(19) United States
(12) Patent Application Publication
(10) Pub. No.: US 2009/0148391 A1
(43) Pub. Date: Jun. 11, 2009

(54) SYNERGISTIC ACTIVE PREPARATIONS COMPRISING DIPHENYLMETHANE DERIVATIVES AND FURTHER SKIN AND/OR HAIR LIGHTENING AND/OR SENILE KERATOSIS REDUCING COMPOUNDS

(75) Inventors: Gerhard Schmaus, Hoxter (DE); Sabine Lange, Holzminden (DE); Gabriele Vielhaber, Holzminden (DE); Karin Schaper, Linnenkamp (DE); Ravikumar Pillai, Emerson, NJ (US)

Correspondence Address: CONNOLLY BOVE LODGE & HUTZ LLP, 1875 EYE STREET, N.W., SUITE 1100, WASHINGTON, DC, 20006, US

(73) Assignee: SYMRISE GmbH & Co. KG, Holzminden (DE)

(21) Appl. No.: 12/159,951

(22) PCT Filed: Jan. 5, 2007
(86) PCT No.: PCT/EP07/50124

§ 371 Date: Oct. 16, 2008

Related U.S. Application Data
(60) Provisional application No. 60/756,205, filed on Jan. 5, 2006.
(60) Provisional application No. 60/804,319, filed on Jun. 9, 2006.

(51) Int. Cl. A61K 8/97; A61K 31/05; A61Q 17/04; A61K 36/48

(52) U.S. Cl. 424/59; 514/731; 424/757; 424/74

(57) ABSTRACT

The present invention relates to specific synergistic active skin- and/or hair-lightening and/or senile keratosis-reducing (cosmetic or pharmaceutical) preparations comprising a mixture comprising or consisting of
a) a tyrosinase-inhibiting amount of one or more compounds of the formula 1:

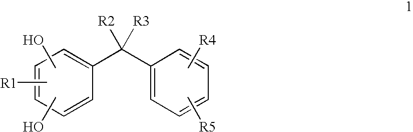

wherein:
   R1 is hydrogen, methyl, straight-chain or branched alkyl having 2-4 C atoms, OH or halogen,
   R2 is hydrogen, methyl or straight-chain or branched alkyl having 2-5 C atoms,
   R3 is methyl or straight-chain or branched alkyl having 2-5 C atoms, and
   R4 and R5 are, independently of one another hydrogen, methyl, straight-chain or branched alkyl having 2-5 C atoms, OH or halogen and
b) a skin- and/or hair lightening and/or senile keratosis-reducing amount of one or more compounds selected from the group consisting of (i) chelating agents, (ii) phenolic derivatives, plant extracts with an amount of phenolic derivatives and (iii) organic acid derivatives as well as uses thereof.

### CROSS REFERENCE TO RELATED APPLICATIONS

This application claims benefit of priority to PCT/EP2007/050124 filed on Jan. 5, 2007, which asserts priority to U.S. Provisional Application Nos. 60/804,319, filed on Jun. 9, 2006, and 60/756,205, filed on Jan. 5, 2006, which are incorporated herein by reference in their entireties.

The present invention relates to specific synergistic active skin- and/or hair-lightening and/or senile keratosis-reducing (cosmetic or pharmaceutical) preparations comprising a mixture comprising or consisting of
a) a tyrosinase-inhibiting amount of one or more compounds of the formula 1:

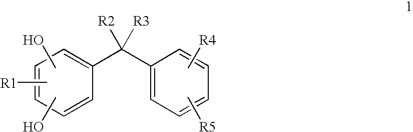

wherein:
   R1 is hydrogen, methyl, straight-chain or branched alkyl having 2-4 C atoms, OH or halogen,
   R2 is hydrogen, methyl or straight-chain or branched alkyl having 2-5 C atoms,
   R3 is methyl or straight-chain or branched alkyl having 2-5 C atoms, and
   R4 and R5 are, independently of one another hydrogen, methyl, straight-chain or branched alkyl having 2-5 C atoms, OH or halogen and
b) a skin- and/or hair lightening and/or senile keratosis-reducing amount of one or more compounds selected from the group consisting of (i) chelating agents, (ii) phenolic derivatives, plant extracts with an amount of phenolic derivatives and (iii) organic acid derivatives.

In the field of the cosmetics industry, there is an increasing demand for agents for lightening the skin and/or hair and/or for agents for reducing senile keratosis. In this context, cosmetics for lightening the skin and/or hair and/or for reducing senile keratosis play a large role above all in Asian countries with a dark-skinned and dark-haired population, but agents for such cosmetic treatments are also gaining in importance in the central European region and in the USA.

The skin and hair colour of humans is substantially determined via the number of melanocytes, and via the melanin concentration and the intensity of melanin biosynthesis, on the one hand intrinsic factors, such as the genetic make-up of an individual, and on the other hand extrinsic factors, such as, in particular, the intensity and frequency of exposure to UV, having a significant influence on skin and hair colour.

Skin- and/or hair-lightening active compounds conventionally intervene in melanin metabolism or catabolism. The melanin pigments, which as a rule are brown to black in colour, are formed in the melanocytes of the skin, transferred into the keratinocytes and cause the colouration of the skin or hair. The brown-black eumelanins are chiefly formed in mammals from hydroxy-substituted aromatic amino acids, such as L-tyrosine and L-DOPA, and the yellow to red phaeomelanins are additionally formed from sulfur-containing molecules (Cosmetics & Toiletries 1996, 111 (5), 43-51). Starting from L-tyrosine, L-3,4-dihydroxyphenylalanine (L-DOPA) is formed by the copper-containing key enzyme tyrosinase, and is in turn converted into dopachrome by tyrosinase. The latter is oxidized to melanin via several steps catalysed by various enzymes.

Skin- and hair-lightening agents are used for various reasons. If the melanin-forming melanocytes are not distributed uniformly in the human skin for whatever reason, pigmental moles which are either lighter or darker than the surrounding areas of skin arise. To eliminate this problem, lightening agents which at least partly help to compensate such pigmental moles are employed. In addition, for many people there is the need to lightening their naturally dark skin colour or to prevent pigmentation of the skin. Very safe and effective skin- and hair-lightening agents are necessary for this. Many skin- and hair-lightening compositions comprise more or less potent tyrosinase inhibitors. However, only one possible route to lightening the skin and hair is taken by this means.

UV-absorbing substances are occasionally also employed for protection against the increase in skin pigmentation induced by UV light. However, this is an effect of purely physical origin and therefore differs from the biological action of skin-lightening agents on cellular melanin formation, which is also detectable in the absence of UV light. In fact, only the UV-induced browning of skin can be prevented by UV filters, whereas a lightening of the skin can also be brought about with biologically active skin lighteners which intervene in melanin biosynthesis.

Hydroquinone, hydroquinone derivatives, such as e.g. arbutin, vitamin C, derivatives of ascorbic acid, such as e.g. ascorbyl palmitate, kojic acid and derivatives of kojic acid, such as e.g. kojic acid dipalmitate, are used in particular in commercially available skin- and hair-lightening compositions.

One of the most frequently used skin- and hair-lightening agents is hydroquinone. However, the substance has a cytotoxic effect on melanocytes and irritates the skin. Such preparations are therefore no longer approved for cosmetic uses e.g. in Europe, Japan and South Africa. Furthermore, hydroquinone is very sensitive to oxidation and can be stabilized in cosmetic preparations only with difficulty.

Vitamin C and ascorbic acid derivatives have only an inadequate action on the skin. They furthermore do not act directly as tyrosinase inhibitors, but reduce the coloured intermediate stages of melanin biosynthesis.

Kojic acid (5-hydroxy-2-hydroxymethyl-4-pyranone) is a tyrosinase inhibitor which, via a chelating of the copper atoms of the enzyme, inhibits the catalytic action thereof; it is employed in commercial skin- and hair-lightening compositions, but has a high sensitizing potential and causes contact allergies.

In the search for novel agents which have a skin- and/or hair-lightening action and/or are active against senile keratosis, efforts are accordingly being made quite generally to discover substances which inhibit the enzyme tyrosinase in the lowest possible concentration, whereby it is furthermore to be ensured that these substances used in cosmetic and/or pharmaceutical products, in addition to having a high activity at the lowest possible concentrations, must also additionally be
toxicologically acceptable,
readily tolerated by the skin,
heat-stable (in particular in the conventional cosmetic and/or pharmaceutical preparations),
preferably odourless and
inexpensive to prepare (i.e. employing standard processes and/or starting from standard precursors).

As disclosed in DE 103 24 566 A1 and WO 2004/105736 A1 diphenylmethane derivatives of the formula 1, preferably compounds of the formula 2, wherein R1 and R3 have the abovementioned meaning, and in particular the styrylresorcinol of the formula 3 (CARN: 85-27-8; 4-(1-phenylethyl)-1,3-dihydroxybenzene)

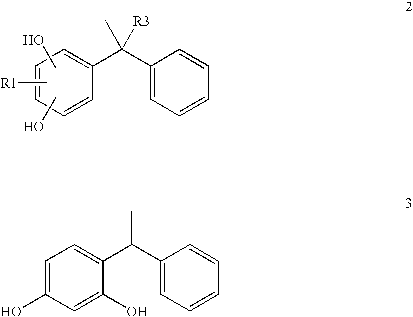

fulfil one or more of the above mentioned requirements.

As further disclosed in DE 103 24 566 A1 and WO 2004/105736 A1 diphenylmethane derivatives of the formula 1, preferably compounds of the formula 2, wherein R1 and R3 have the abovementioned meaning, and in particular the styrylresorcinol of the formula 3 have a good skin and/or hair lightening activity and/or a good activity for reducing senile keratosis. As also disclosed in DE 103 24 566 A1 and WO 2004/105736 A1 the activity in skin and/or hair lightening and/or reducing senile keratosis is based on the ability to inhibit the enzyme tyrosinase, the key enzyme in the production of melanin. This tyrosinase-inhibiting activity has been clearly demonstrated in in-vitro enzyme assays on 3T3 firbrosarcoma cells or B16V mouse melanoma cells.

In the search for new and improved methods for skin and/or hair lightening and/or reducing senile keratosis the aim of a person skilled in the art is on the one hand to fully substitute commercially available products like hydroquinone, kojic acid, vitamin C or derivatives thereof, which show, in particular in a high concentrations, undesirable side effects, with other skin and/or hair lightening and/or senile keratosis reducing agents. On the other hand it is conceivable to reduce the concentration of the commercially available agents with the undesirable side effects and combine these agents with other agents, which show less side effects and are more compatible. A synergistic combination of a commercially available agent with side effects with one or more other agents with less side effect would be ideal.

Thus, surprisingly a cosmetic and/or pharmaceutical preparation, comprising a mixture comprising or consisting of
a) a tyrosinase-inhibiting amount of one or more compounds of the formula 1:

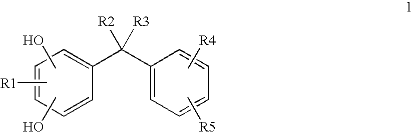

wherein:
   R1 is hydrogen, methyl, straight-chain or branched alkyl having 2-4 C atoms, OH or halogen,
   R2 is hydrogen, methyl or straight-chain or branched alkyl having 2-5 C atoms,
   R3 is methyl or straight-chain or branched alkyl having 2-5 C atoms, and
   R4 and R5 are, independently of one another hydrogen, methyl, straight-chain or branched alkyl having 2-5 C atoms, OH or halogen and
b) a skin- and/or hair lightening and/or senile keratosis-reducing amount of one or more compounds selected from the group consisting of (i) chelating agents, (ii) phenolic derivatives, plant extracts with an amount of phenolic derivatives and (iii) organic acid compounds
   fulfils the requirements of the present invention, as the preparation according to the invention comprises a synergistic combination of the different skin and/or hair lightening and/or senile keratosis reducing agents of constituents a) and b).

In this context, the substituents OH, R1, R4 and R5 can in each case occupy (as indicated by the drawing) any desired position on the particular aromatic ring (ortho, meta or para to the bridge between the rings).

A further embodiment of the present invention is the use of a mixture comprising or consisting of
a) a tyrosinase-inhibiting amount of one or more compounds of the formula 1:

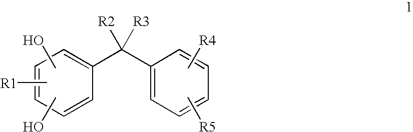

wherein:
   R1 is hydrogen, methyl, straight-chain or branched alkyl having 2-4 C atoms, OH or halogen,
   R2 is hydrogen, methyl or straight-chain or branched alkyl having 2-5 C atoms,
   R3 is methyl or straight-chain or branched alkyl having 2-5 C atoms, and
   R4 and R5 are, independently of one another hydrogen, methyl, straight-chain or branched alkyl having 2-5 C atoms, OH or halogen and
b) a skin- and/or hair lightening and/or senile keratosis-reducing amount of one or more compounds selected from the group consisting of (i) chelating agents, (ii) phenolic derivatives, plant extracts with an amount of phenolic derivatives and (iii) organic acid derivatives
   in the manufacture of a cosmetic and/or pharmaceutical preparation for lightening skin and/or reducing senile keratosis.

Another embodiment of the present invention is a use of a preparation according to the present invention as described hereinbefore.

A still further embodiment of the present invention is a method for lightening skin and/or hair and/or for reducing senile keratosis, comprising or consisting of the step:
c) application of a preparation according to any of claims 1 to 11 to skin and/or hair.

A yet still further embodiment of the present invention is a process for the production of a preparation for lightening skin and/or hair and/or for reducing senile keratosis, comprising or consisting of the following steps:
d) providing one or more compounds of the formula 1

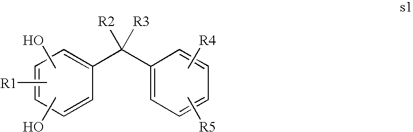

wherein:
   R1 is hydrogen, methyl, straight-chain or branched alkyl having 2-4 C atoms, OH or halogen,
   R2 is hydrogen, methyl or straight-chain or branched alkyl having 2-5 C atoms,
   R3 is methyl or straight-chain or branched alkyl having 2-5 C atoms, and
   R4 and R5 are, independently of one another hydrogen, methyl, straight-chain or branched alkyl having 2-5 C atoms, OH or halogen,
e) providing one or more compounds selected from the group of (i) chelating agents, (ii) phenolic derivatives, plant extracts with an amount of phenolic derivatives and (iii) organic acid derivatives,
f) providing one or more further compounds and
   mixing one or more compounds provided in step d) and one or more compounds provided in step e) together with one or more compounds provided in step f) to form a preparation according to the invention as described hereinbefore.

The following preferred aspects are relevant for each embodiment of the present invention.

Particularly preferred phenolic compounds of the formula 1 are those of the formula 2, wherein R1 and R3 have the abovementioned meaning, and in particular preferred is the styrylresorcinol of the formula 3 (CARN: 85-27-8; 4-(1-phenylethyl)-1,3-dihydroxybenzene)):

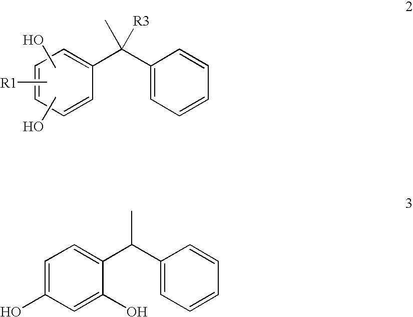

Preferred embodiments of the preparations which are preferred according to the invention and uses thereof are described in the following and in the examples and the claims. The synergistic activity of the preparations according to the invention is further shown in table 1.

TABLE 1
Preferred use level ranges and use level ratios of different types of skin
and/or hair lightening and/or senile keratosis reducing agents like (i) chelating
agents or (ii) phenolic derivatives and plant extracts comprising phenolic
derivatives or (iii) organic acid derivatives used in combination with 4-(1-
phenylethyl)-1,3,dihydroxybenzene (CARN: 85-25-8; with preferred use level range
of 0.1% to 2%) in finished cosmetic and pharmaceutical, in particular
dermatological preparations for skin and/or hair lightening and/or for the treatment
of senile keratosis.

| | use level | use level ratio | preferred use level ratio |
|---|---|---|---|
| | range [weight | chelating agent:CARN | chelating agent:CARN |
| (i) Chelating Agents | %] | 85-27-8 | 85-27-8 |
| Kojic acid | 0.5-5 | 1:4 to 50:1 | 1:2 to 20:1 |
| EDTA | 0.02-0.5 | 1:100 to 5:1 | 1:50 to 2:1 |
| Hinokitiol | 0.1:1 | 1:20 to 10:1 | 1:10 to 5:1 |
| Tropolone | 0.05-1 | 1:40 to 10:1 | 1:20 to 5:1 |
| Ascorbic acid | 0.5-5 | 1:4 to 50:1 | 1:2 to 20.1 |
| Lactic acid | 0.2-5 | 1:10 to 50:1 | 1:5 to 20:1 |
| Salicylic acid | 0.2-5 | 1:10 to 50:1 | 1:5 to 20:1 |
| Glycolic acid | 0.2-5 | 1:10 to 50:1 | 1:5 to 20:1 |
| Citric acid | 0.2-5 | 1:10 to 50:1 | 1:5 to 20:1 |
| Malic acid | 0.2-5 | 1:10 to 50:1 | 1:5 to 20:1 |

| (ii) Phenolic | | | |
|---|---|---|---|
| derivatives and | | | |
| plant extracts | preferred use | use level ratio | preferred use level ratio |
| comprising phenolic | level range | phenolic compound:CARN | phenolic compound:CARN |
| derivatives | [weight %] | 85-27-8 | 85-27-8 |
| Arbutine | 0.5-5 | 1:4 to 50:1 | 1:2 to 20:1 |
| Hydroquinone | 0.2-4 | 1:10 to 40.1 | 1:5 to 20:1 |
| Resorcinol | 0.5-5 | 1:4 to 50:1 | 1:2 to 20:1 |
| 4-Butylresorcinol | 0.5-5 | 1:4 to 50:1 | 1:2 to 20:1 |
| Bearberry extract | 0.5-5 | 1:4 to 50:1 | 1:2 to 20:1 |
| Pinus extract | 0.5-5 | 1:4 to 50:1 | 1:2 to 20:1 |
| Mulberry extract | 0.5-5 | 1:4 to 50:1 | 1:2 to 20:1 |
| Soybean extract | 0.5-5 | 1:4 to 50:1 | 1:2 to 20:1 |
| Artocarpus extract | 0.5-5 | 1:4 to 50:1 | 1:2 to 20:1 |
| Licorice extract | 0.5-5 | 1:4 to 50:1 | 1:2 to 20:1 |

| | | use level ratio | |
|---|---|---|---|
| | preferred use | organic acid | preferred use level ratio |
| (iii) organic acid | level range | derivative:CARN | organic acid derivative:CARN |
| derivatives | [weight %] | 85-27-8 | 85-27-8 |
| Azelaic acid | 0.5-5 | 1:4 to 50:1 | 1:2 to 20:1 |
| 9-Octadeceneoic acid | 0.5-5 | 1:4 to 50:1 | 1:2 to 20:1 |
| alpha-Lipoic acid | 0.2-1 | 1:10 to 10:1 | 1:5 to 5:1 |
| Retinoic acid | 0.01-0.1 | 1:200 to 1:1 | 1:50 to 2:1 |
| Niacinamide | 0.5-5 | 1:4 to 50:1 | 1:2 to 20:1 |
| Undecylenoyl | 0.5-5 | 1:4 to 50:1 | 1:2 to 20:1 |
| Phenylalanine | | | |

In this context, it is shown that the common agents of constituent b), in particular as disclosed in table 1, which have a skin and/or hair lightening and/or senile keratosis reducing activity, are selected from the group consisting of
(i) chelating agents, preferably kojic acid, EDTA, hinokitiol, tropolone, ascorbic acid, lactic acid, salicylic acid, glycolic acid, citric acid and malic acid,
(ii) phenolic derivatives and plant extracts comprising an amount of phenolic derivatives, preferably arbutin, hydroquinone, resorcinol, 4-butyl resorcinol, bearberry extract (Arctostaphylos uva-ursi), pinus extract (Pinus sylvestris), Mulberry extract (Morus alba), soybean extract (Glycine max.), artocarpus extract (Artocarpus incisus) and licorice extract (Glycyrrhiza glabra) and/or
(iii) organic acid derivatives, preferably azelaic acid, 9-octadecenoic acid, alpha lipoic acid, retinoic acid, niacinamide and undecylenoyl phenylalanine
   have in combination with diphenylmethane derivatives of the formula 1, preferably compounds of the formula 2, wherein R1 and R3 have the abovementioned meaning, and in particular preferably the styrylresorcinol of the formula 3 (CARN: 85-27-8; 4-(1-phenylethyl)-1,3-dihydroxybenzene), in particular with the concentrations and weight ratio ranges, as described in more detail in table 1, a synergistic improved activity on skin and/or hair lightening and/or reducing of senile keratosis.

The following agents selected from the group consisting of
(i) chelating agents selected from the group consisting of kojic acid, EDTA, ascorbic acid, lactic acid and salicylic acid and/or
(ii) phenolic derivatives selected from the group consisting of arbutin, hydroquinone and 4-butyl resorcinol, and plant extracts selected from the group consisting of bearberry extract (Arctostaphylos uva-ursi), Mulberry extract (Morus alba), artocarpus extract (Artocarpus incisus) and licorice extract (Glycyrrhiza glabra) and/or
(iii) organic acid derivatives selected from the group consisting of azelaic acid, 9-octadecenoic acid, niacinamide and undecylenoyl phenylalanine
   have in combination with diphenylmethane derivatives of the formula 1, preferably compounds of the formula 2, wherein R1 and R3 have the abovementioned meaning, and in particular preferably the styrylresorcinol of the formula 3 (CARN: 85-27-8; 4-(1-phenylethyl)-1,3-dihydroxybenzene), in particular with the concentrations and weight ratio ranges, as described in more detail in table 1, a synergistic improved activity on skin and/or hair lightening and/or reducing of senile keratosis.

The strongest synergistic effects are received with styrylresorcinol of the formula 3 in cosmetic or pharmaceutical, in particular dermatological preparations at a concentration from 0, 1 to 2 wt % based on the weight of the finished preparation.

In a preferred embodiment the preparation according to the invention including the preferred embodiments for constituents (a) and/or (b) does not consist of the preparations as described in tables 6 and 7 below.

In a further preferred embodiment the preparation according to the invention including the preferred embodiments for constituents (a) and/or (b) does not comprise a content of an oily phase between 0.05 to 12 wt. % based on the finished preparation.

The reason for the synergistic activity between compounds of general formula 1 and compounds selected from the group selected of (i) chelating agents, (ii) phenolic derivatives, plant extracts with an amount of phenolic derivatives and (iii) organic acid derivatives has not been clearly identified yet. It may be of very different origins, whereby it seems that some biologic mechanisms may still not have been identified yet. However, at the moment the following explanation seem plausible:
Diphenylmethane derivatives of the formula 1, preferably compounds of the formula 2, wherein R1 and R3 have the above-mentioned meaning, and in particular the styrylresorcinol of the formula 3 have anti-oxidative and therefore product stabilizing properties, which prevents or slows down the decomposition of skin and/or hair lightening and/or senile keratosis reducing compounds of constituent b) of the preparation, which may be sensitive against UV-, temperature or pH-influence. Thus, the efficiency of these compounds in the preparation may be improved.
Diphenylmethane derivatives of the formula 1, preferably compounds of the formula 2, wherein R1 and R3 have the above-mentioned meaning, and in particular the styrylresorcinol of the formula 3 have a skin and/or hair lightening and/or senile keratosis reducing activity, which is based on the reversible blockade inhibition of the active centre of the enzyme tyrosinase.

Moreover, further biological mechanisms are described in the context of diphenylmethane derivatives of formula 1, in particular styrylresocrinol of formula 3, which may have an impact on the activity of skin and/or hair lightening and/or senile keratosis reduction. For example, for the chelating agent kojic acid the activity of skin and/or hair lightening and/or senile keratosis reduction is related to chelating of the copper atoms of the enzyme tyrosinase. For other skin and/or hair lightening and/or senile keratosis reducing agents, for example 9-octadecenoic, acid it is described that the effect is based on the inhibition of the transfer of the melanosomes in melanocytes to keratinocytes. Thus, the synergistic activity of the combination of compounds of formula 1 and compounds selected from the group consisting of (i) chelating agents, (ii) phenolic derivatives, plant extracts with an amount of phenolic derivatives and (iii) organic acid derivatives can be based on more than one effects.
Theoretically the synergistically improvement of the activity of the combination of compounds of formula 1 and compounds selected from the group consisting of (i) chelating agents, (ii) phenolic derivatives, plant extracts with an amount of phenolic derivatives and (iii) organic acid derivatives can be based on penetration-inducing properties of the diphenylmethane derivatives of formula 1, however, this is at the moment a mere speculative reasoning.

In the context of this invention the diphenylmethane derivatives of the formula 1, preferably compounds of the formula 2, wherein R1 and R3 have the abovementioned meaning, and in particular the styrylresorcinol of the formula 3 described in more detail in the following (CARN: 85-27-8; 4-(1-phenylethyl)-1,3-dihydroxybenzene) can be incorporated without problems into preparations according to the invention.

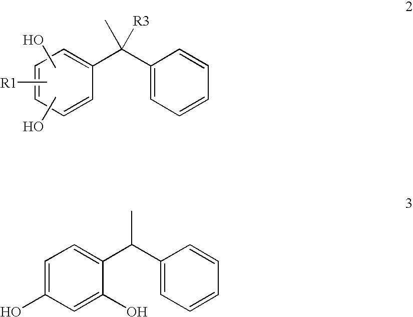

The particularly suitable preparations with synergistic activity according to the invention are chiefly used according to the invention for cosmetic reasons, but in exceptional cases they can also have a therapeutic character.

In this context, the concentration of the diphenylmethane derivatives of the formula 1, preferably compounds of the formula 2, wherein R1 and R3 have the abovementioned meaning, and in particular the styrylresorcinol of the formula 3 in the finished preparations according to the invention, in particular to be applied topically, is preferably in the range of from 0.001 to 6 wt. %, preferably in the range of from 0.01 to 4 wt. % and particularly preferably in the range of from 0.01 to 2 wt. %. The tyrosinase-inhibiting active compound can be employed here (a) prophylactically or (b) as required.

The concentration of the amount of active compound to be applied e.g. daily varies and depends on the physiological state of the subject and individual-specific parameters, such as age or body weight.

It is to be pointed out that the term diphenylmethane derivatives in the context of the present invention also includes, in the case of the derivatives of the formula 1 which have differently substituted phenyl radicals and for which at the same time R2 and R3 are different, the pure S-configured enantiomers, the R-configured enantiomers and any desired mixtures of S- and R-configured enantiomers. For commercial reasons, it is indeed particularly advantageous in these cases to employ mixtures of racemates of the particular diphenylmethane derivatives for lightening skin and/or hair and/or for reducing senile keratosis, since these are particularly readily accessible by synthesis, but the pure enantiomers or non-racemic mixtures of these enantiomers are likewise suitable for the purposes according to the invention.

In this context, the mixture comprising or consisting of (a) one or more compounds of formula 1 and (b) one or more compounds selected from the group consisting of (i) chelating agents, (ii) phenolic derivatives, plant extracts with an amount of phenolic derivatives and (iii) organic acid derivatives used according to the invention including the preferred embodiments of the constituents (a) and/or (b) can be incorporated without difficulties into the chiefly aqueous cosmetic or pharmaceutical, in particular dermatological preparations according to the invention, such as, inter alia, pump sprays, aerosol sprays, creams, ointments, tinctures, lotions and specific nail care products and the like. It is also possible, and in some cases advantageous, to combine the mixture comprising or consisting of (a) one or more compounds of formula 1 and (b) one or more compounds selected from the group consisting of (i) chelating agents, (ii) phenolic derivatives, plant extracts with an amount of phenolic derivatives and (iii) organic acid derivatives including the preferred embodiments of constituents (a) and/or (b) with further active ingredients. The cosmetic and/or pharmaceutical, in particular dermatological/keratological preparations according to the invention comprising the mixture comprising or consisting of (a) one or more compounds of formula 1 and (b) one or more compounds selected from the group consisting of (i) chelating agents, (ii) phenolic derivatives, plant extracts with an amount of phenolic derivatives and (iii) organic acid derivatives including the preferred embodiments of constituents (a) and/or (b) used according to the invention can have the conventional auxiliary compounds and additives (base ingredients) and serve for the treatment of skin and/or hair in the sense of a pharmaceutical, in particular dermatological or keratological treatment or a treatment in the sense of care cosmetics. However, they can also be employed in decorative cosmetics.

The significant synergistic activity has been found for preparations comprising the mixture comprising or consisting of (a) one or more compounds of formula 1 and (b) one or more compounds selected from the group consisting of (i) chelating agents, (ii) phenolic derivatives, plant extracts with an amount of phenolic derivatives and (iii) organic acid derivatives including the preferred embodiments of constituents (a) and/or (b) used according to the invention in which the content and the weight ration of constituents (a) and (b) are based on the total weight of the preparation as shown in table 1 above.

The synergistic active cosmetic or pharmaceutical, in particular dermatological preparations which comprise a mixture comprising or consisting of (a) one or more compounds of formula 1 and (b) one or more compounds selected from the group consisting of (i) chelating agents, (ii) phenolic derivatives, plant extracts with an amount of phenolic derivatives and (iii) organic acid derivatives including the preferred embodiments of constituents (a) and/or (b) used according to the invention are preferably in the form of an O/W emulsion.

A preparation according to the invention, in particular in the form of an O/W emulsion, including the preferred embodiments of constituents (a) and/or (b) regularly comprises one or more of the following solvents: water or aqueous (salt) solutions, alcohols, diols or polyols of low C number (preferably having 2 to 6 C atoms, specifically having 2 to 4 C atoms), and ethers thereof, preferably ethanol, isopropanol, propylene glycol (1,2-propanediol), glycerol, ethylene glycol, ethylene glycol monoethyl or monobutyl ether, propylene glycol monomethyl, monoethyl or monobutyl ether, diethylene glycol monomethyl or monoethyl ether and analogous products. Mixtures of the abovementioned solvents are used in particular. In the case of alcoholic solvents, water can be a further constituent.

Further conventional cosmetic auxiliary substances and additives (including water) can be present in amounts of 5-99 wt. %, preferably 10-90 wt. %, based on the total weight of the preparation.

A preparation according to the invention including the preferred embodiments of constituents (a) and/or (b), in particular in the form of an O/W emulsion, regularly comprises one or more of the following thickeners, which can advantageously be chosen from the group consisting of silicon dioxide, aluminium silicates, polysaccharides or derivatives thereof, e.g. hyaluronic acid, xanthan gum, hydroxypropyl-methylcellulose, particularly advantageously from the group consisting of polyacrylates, preferably a polyacrylate from the group consisting of the so-called Carbopols, for example Carbopols of types 980, 981, 1382, 2984, 5984, in each case individually or in combination.

Preparations according to the invention in the form of an O/W emulsion including the preferred embodiments of constituents (a) and/or (b) advantageously comprise one or more emulsifiers.

O/W emulsifiers are advantageously chosen from the group consisting of polyethoxylated or polypropoxylated or polyethoxylated and polypropoxylated products, e.g.:
the fatty alcohol ethoxylates
the ethoxylated wool wax alcohols,
the polyethylene glycol ethers of the general formula

R—O—(—CH₂—CH₂—O—)ₙ—R′,
the fatty acid ethoxylates of the general formula

R—COO—(—CH₂—CH₂—O—)ₙ—H,
the etherified fatty acid ethoxylates of the general formula

R—COO—(—CH₂—CH₂—O—)ₙ—R′,
the esterified fatty acid ethoxylates of the general formula

R—COO—(—CH₂—CH₂—O—)ₙ—C(O)—R′,
the polyethylene glycol glycerol fatty acid esters
the ethoxylated sorbitan esters
the cholesterol ethoxylates
the ethoxylated triglycerides
the alkyl ether-carboxylic acids of the general formula

R—COO—(—CH₂—CH₂—O—)ₙ—OOH, wherein n represents a number from 5 to 30,
the polyoxyethylene sorbitol fatty acid esters
the alkyl ether-sulfates of the general formula

R—O—(—CH₂—CH₂—O—)ₙ—SO₃—H
the fatty alcohol propoxylates of the general formula

R—O—(—CH₂—CH(CH₃)—O—)ₙ—H
the polypropylene glycol ethers of the general formula

R—O—(—CH₂—CH(CH₃)—O—)ₙ—R′
the propoxylated wool wax alcohols,
the etherified fatty acid propoxylates

R—COO—(—CH₂—CH(CH₃)—O—)ₙ—R′
the esterified fatty acid propoxylates of the general formula

R—COO—(—CH₂—CH(CH₃)—O—)ₙ—C(O)—R′
the fatty acid propoxylates of the general formula

R—COO—(—CH₂—CH(CH₃)—O—)ₙ—H,
the polypropylene glycol glycerol fatty acid esters
the propoxylated sorbitan esters
the cholesterol propoxylates
the propoxylated triglycerides
the alkyl ether-carboxylic acids of the general formula

R—O—(—CH₂—CH(CH₃)—O—)ₙ—CH₂—COOH,
the alkyl ether-sulfates and the acids on which these sulfates are based of the general formula

R—O—(—CH₂—CH(CH₃)—O—)ₙ—SO₃—H,
the fatty alcohol ethoxylates/propoxylates of the general formula

R—O—Xₙ—Yₘ—H
the polypropylene glycol ethers of the general formula

R—O—Xₙ—Yₘ—R′
the etherified fatty acid propoxylates of the general formula

R—COO—Xₙ—Yₙ—R′
the fatty acid ethoxylates/propoxylates of the general formula

R—COO—Xₙ—Yₘ—H.

According to the invention, the polyethoxylated or polypropoxylated or polyethoxylated and polypropoxylated O/W emulsifiers employed are particularly advantageously chosen from the group consisting of substances having HLB values of 11-18, very particularly advantageously having HLB values of 14.5-15.5, if the O/W emulsifiers contain saturated radicals R and R′. If the O/W emulsifiers contain unsaturated radicals R and/or R′, or isoalkyl derivatives are present, the preferred HLB value of such emulsifiers can also be lower or higher.

It is of advantage to choose the fatty alcohol ethoxylates from the group consisting of ethoxylated stearyl alcohols, cetyl alcohols and cetylstearyl alcohols (cetearyl alcohols). The following are particularly preferred:

polyethylene glycol (n) stearyl ether (steareth-n), where n=13-20,
polyethylene glycol (n) cetyl ether (ceteth-n), where n=13-20,
polyethylene glycol (n) isocetyl ether (isoceteth-n), where n=13-20,
polyethylene glycol (n) cetylstearyl ether (ceteareth-n), where n=13-20,
polyethylene glycol (m) isostearyl ether (isosteareth-m), where m=12-20
polyethylene glycol (k) oleyl ether (oleth-k), where k=12-15
polyethylene glycol (12) lauryl ether (laureth-12),
polyethylene glycol (12) isolauryl ether (isolaureth-12).

It is furthermore advantageous to chose the fatty acid ethoxylates from the following group:

polyethylene glycol (n) stearate, where n=20-25
polyethylene glycol (m) isostearate, where m=12-25
polyethylene glycol (k) oleate, where k=12-20

Sodium laureth-11 carboxylate can advantageously be used as an ethoxylated alkyl ether-carboxylic acid or salt thereof. Sodium laureth 1-4 sulfate can advantageously be used as an alkyl ether-sulfate. Polyethylene glycol (30) cholesteryl ether can advantageously be used as an ethoxylated cholesterol derivative. Polyethylene glycol (25) soyasterol has also proved suitable.

The polyethylene glycol (60) evening primrose glycerides can advantageously be used as ethoxylated triglycerides.

It is furthermore advantageous to chose the polyethylene glycol glycerol fatty acid esters from the group consisting of

polyethylene glycol (20-23) glyceryl-laurate polyethylene glycol (6) glyceryl-caprylate/caproate, polyethylene glycol (20) glyceryl-oleate, polyethylene glycol (20) glyceryl-isostearate, polyethylene glycol (18) glyceryl-oleate/cocoate.

It is likewise favourable to choose the sorbitan esters from the group consisting of polyethylene glycol (20) sorbitan monolaurate, polyethylene glycol (20) sorbitan monostearate, polyethylene glycol (20) sorbitan monoisostearate, polyethylene glycol (20) sorbitan monopalmitate and polyethylene glycol (20) sorbitan monooleate.

The (in particular topical) cosmetic or pharmaceutical, in particular dermatological preparations according to the invention, in particular skin- and/or hair-lightening compositions, including the preferred embodiments of constituents (a) and/or (b) can comprise cosmetic auxiliary substances and additives such as are conventionally used in such preparations, e.g. sunscreen agents, preservatives, bactericides, fungicides, virucides, cooling active compounds, insect repellents (e.g. DEET, IR 3225, Dragorepel), plant extracts, antiinflammatory active compounds, substance which accelerate wound healing (e.g. chitin or chitosan and derivatives thereof), film-forming substances (e.g. polyvinylpyrrolidones or chitosan or derivatives thereof), the usual antioxidants, vitamins (e.g. vitamin C derivatives, tocopherols and derivatives, vitamin A and derivatives), skin care agents (e.g. cholesterol, ceramides, pseuodceramides), softening, moisturizing and/or humectant substances (in particular glycerol, urea or 1,2-alkanediols, such as 1,2-pentanediol, 1,2-hexanediol and/or 1,2-octanediol), saturated fatty acids, mono- or polyunsaturated fatty acids, alpha-hydroxy acids, polyhydroxy-fatty acids or derivatives thereof (e.g. linoleic acid, alpha-linolenic acid, gamma-linolenic acid or arachidonic acid and the particular natural or synthetic esters thereof, waxes or other conventional constituents of a cosmetic or dermatological preparation, such as alcohols, polyols, polymers, foam stabilizers, electrolytes, organic solvents, silicone derivatives, antidandruff active compounds (e.g. climbazole, ketoconazole, piroctonoleamine, zinc pyrithione), hair care agents, perfume, substances for preventing foaming, dyestuffs, pigments which have a colouring action, thickening agents, surface-active substances, surfactants, emulsifiers, plant parts and plant extracts (e.g. arnica, aloe, beard lichen, ivy, stinging nettle, ginseng, henna, camomile, marigold, rosemary, sage, blackberry, horsetail or thyme), royal jelly, propolis, proteins, protein hydrolysates, yeast extracts, hop and wheat extracts, peptides or thymus extracts.

The particular amounts of cosmetic or dermatological auxiliary substances and additives and of one or more odoriferous substances (perfumes) to be employed can be easily be determined according to the nature of the particular product by simple trials by the person skilled in the art.

The preparations according to the invention which comprise a mixture comprising or consisting of (a) one or more compounds of formula 1 and (b) one or more compounds selected from the group consisting of (i) chelating agents, (ii) phenolic derivatives, plant extracts with an amount of phenolic derivatives and (iii) organic acid derivatives including the preferred embodiments for constituents (a) and/or (b) used according to the invention can also comprise further active compounds having a skin- and/or hair lightening and/or senile keratosis reducing action which have not been described above. According to the invention, all the further skin-lightening active compounds which are suitable or usual for cosmetic and/or pharmaceutical, in particular dermatological uses can be used here. Advantageous skin-lightening active compounds in this respect are kojic acid (5-hydroxy-2-hydroxymethyl-4-pyranone), kojic acid dipalmitate, ascorbic acid derivatives, hydroquinone derivatives, sulfur-containing molecules, such as e.g. glutathione or cysteine, alpha-hydroxy acids (e.g. citric acid, lactic acid, malic acid) and derivatives thereof, N-acetyl-tyrosine and derivatives, gluconic acid, further 4-alkylresorcinol derivatives which have not been described in group (ii) above, such as 4-(1-phenylethyl)-1,3-benzenediol, chromone derivatives, such as aloesin, flavonoids, thymol derivatives, 1-aminoethylphosphinic acid, thiourea derivatives, ellagic acid, nicotinamide, zinc salts, such as e.g. zinc chloride or gluconate, thujaplicin and derivatives, triterpenes, such as maslic acid, sterols, such as ergosterol, benzofuranones, such as senkyunolide, vinyl- and ethylguaiacol, further dionic acids, inhibitors of nitrogen oxide synthesis, such as e.g. L-nitroarginine and derivatives thereof 2,7-dinitroindazole or thiocitrulline, metal chelators (e.g. palmitic acid, phytic acid, lactoferrin, humic acid, bile acid, bile extracts, bilirubin, biliverdin, 1,1,4,77-diethylenetriaminepentaacetic acid, EGTA and derivatives thereof, further retinoid derivatives, soya milk and extract, serine protease inhibitors or liponic acid or other synthetic or natural active compounds for lightening of the skin and hair, the latter also being used in the form of an extract from plants, such as e.g. rice extract, papaya extract or constituents concentrated therefrom, such as glabridin or licochalcone A, Artocarpus extract, extract from Rumex and Ramulus species and extracts from Vitis species or stilbene derivatives concentrated therefrom, extract from Saxifraga, mulberry, Scutelleria or/and grape.

For use, the cosmetically and/or pharmaceutically, in particular dermatologically active preparations according to the invention comprising a mixture comprising or consisting of (a) one or more compounds of formula 1 and (b) one or more compounds selected from the group consisting of (i) chelating agents, (ii) phenolic derivatives, plant extracts with an amount of phenolic derivatives and (iii) organic acid derivatives including the preferred embodiments for constituents (a) and/or (b) are applied to the skin and/or hair in a sufficient amount in the conventional manner for cosmetics and pharmaceutical, in particular dermatics. In this context, those cosmetic and pharmaceutical, in particular dermatological preparations which additionally comprise one or more sunscreen filters (UV absorbers, UV filters) and thereby act both as hair- or skin-lightening or senile keratosis-reducing compositions and as sunscreen compositions offer particular advantages.

Preparations according to the invention including the preferred embodiments for constituents (a) and/or (b) comprising one or more sunscreen filters (UV absorbers) preferably have a total content of UV absorbers in the range of from 0.1 to 30 wt. %, preferably in the range of from 0.2 to 20 wt. %, in particular 0.5 to 15 wt. %, based on the total weight of the preparation.

The preparations according to the invention including the preferred embodiments for constituents (a) and/or (b) advantageously comprise at least one UV-A filter and/or at least one UV-B filter and/or a broadband filter and/or at least one inorganic pigment. Preparations according to the invention preferably comprise at least one UV-B filter or one broadband filter, and furthermore preferably at least one UV-A filter and at least one UV-B filter.

Suitable sunscreen agents (UV absorbers) are e.g. organic UV absorbers from the class consisting of 4-aminobenzoic acid and derivatives, salicylic acid derivatives, benzophenone derivatives, dibenzoylmethane derivatives, diphenyl acrylates, 3-imidazol-4-yl-acrylic acid and esters thereof, benzofuran derivatives, benzylidenemalonate derivatives, polymeric UV absorbers, containing one or more organosilicon radicals, cinnamic acid derivatives, camphor derivatives, trianilino-s-triazine derivatives, 2-hydroxyphenylbenzotriazole derivatives, phenylbenzimidazolesulfonic acid derivatives and salts thereof, anthranilic acid menthyl ester, benzotriazole derivatives, indole derivatives.

The UV absorbers mentioned below, which can be employed in the context of the present invention, are preferred, but of course not limiting.

Advantageous UV filters are

UV-B filters, such as e.g.:
p-aminobenzoic acid
p-aminobenzoic acid ethyl ester (25 mol) ethoxylated
p-dimethylaminobenzoic acid 2-ethylhexyl ester
p-aminobenzoic acid ethyl ester (2 mol) N-propoxylated
p-aminobenzoic acid glycerol ester
salicylic acid homomethyl ester (homosalate) (Neo-Heliopan®HMS)
salicylic acid 2-ethylhexyl ester (Neo-Heliopan®OS)
triethanolamine salicylate
4-isopropylbenzyl salicylate
anthranilic acid menthyl ester (Neo Heliopan®MA)
diisopropylcinnamic acid ethyl ester
p-Methoxycinnamic acid 2-ethylhexyl ester (Neo Heliopan®AV)
diisopropylcinnamic acid methyl ester
p-methoxycinnamic acid isoamyl ester (Neo Heliopan®E 1000)
p-methoxycinnamic acid diethanolamine salt
p-methoxycinnamic acid isopropyl ester
2-phenylbenzimidazolesulfonic acid and salts (Neo Heliopan®Hydro)
3-(4′-trimethylammonium)-benzylidene-bornan-2-one methyl sulfate
β-imidazole-4(5)-acrylic acid (urocanic acid)
3-(4′-sulfo)benzylidene-bornan-2-one and salts
3-(4′-methylbenzylidene)-d,l-camphor (Neo Heliopan®MBC)
3-benzylidene-d,l-camphor
N-[(2 and 4)-[2-(oxoborn-3-ylidene)methyl]benzyl]-acrylamide polymer
4,4′-[(6-[4-(1,1-dimethyl)-aminocarbonyl)-phenylamino]-1,3,5-triazine-2,4-diyl)diimino]-bis-(benzoic acid 2-ethylhexyl ester) (Uvasorb®HEB)
benzylidenemalonate-polysiloxane (Parsol®SLX)
glyceryl ethylhexanoate dimethoxycinnamate
dipropylene glycol salicylate
tris(2-ethylhexyl) 4,4′,4″-(1,3,5-triazine-2,4,6-triyltriimino)tribenzoate (Uvinul®T150)
   broadband filters, such as e.g.:
2-ethylhexyl 2-cyano-3,3-diphenylacrylate (Neo Heliopan®303)
ethyl 2-cyano-3,3′-diphenylacrylate
2-hydroxy-4-methoxybenzophenone (Neo Heliopan®BB)
2-hydroxy-4-methoxybenzophenone-5-sulfonic acid
dihydroxy-4-methoxybenzophenone
2,4-dihydroxybenzophenone
tetrahydroxybenzophenone
2,2′-dihydroxy-4,4′-dimethoxybenzophenone
2-hydroxy-4-n-octoxybenzophenone
2-hydroxy-4-methoxy-4′-methylbenzophenone
sodium hydroxymethoxybenzophenone sulfonate
disodium 2,2′-dihydroxy-4,4′-dimethoxy-5,5′-disulfo-benzophenone
phenol, -(2H-benzotriazol-2-yl)-4-methyl-6-(2-methyl-3-(1,3,3,3-tetramethyl-1-(trimethylsilyl)-oxy)-disiloxyanyl)-propyl), (Mexoryl®XL)
2,2′-methylene-bis-(6-(2H-benztriazol-2-yl)-4-(1,1,3,3-tetramethylbutyl)-phenol), (Tinosorb®M)
2,4-bis-[4-(2-ethylhexyloxy)-2-hydroxyphenyl]-1,3,5-triazine
2,4-bis-[{(4-(2-ethyl-hexyloxy)-2-hydroxy}-phenyl]-6-(4-methoxyphenyl)-1,3,5-triazine, (Tinosorb®S)
2,4-bis-[{(4-(3-sulfonato)-2-hydroxy-propyloxy)-2-hydroxy}-phenyl]-6-(4-methoxyphenyl)-1,3,5-triazine sodium salt
2,4-bis-[{(3-(2-propyloxy)-2-hydroxy-propyloxy)-2-hydroxy}-phenyl]-6-(4-methoxy-phenyl)-1,3,5-triazine
2,4-bis-[{4-(2-ethyl-hexyloxy)-2-hydroxy}-phenyl]-6-[4-(2-methoxyethyl-carbonyl)-phenylamino]-1,3,5-triazine
2,4-bis-[{4-(3-(2-propyloxy)-2-hydroxy-propyloxy)-2-hydroxy}-phenyl]-6-[4-(2-ethylcarboxyl)-phenylamino]-1,3,5-triazine
2,4-bis-[{4-(2-ethyl-hexyloxy)-2-hydroxy}-phenyl]-6-(1-methyl-pyrrol-2-yl-)-1,3,5-triazine
2,4-bis-[{4-tris-(trimethylsiloxy-silylpropyloxy)-2-hydroxy}-phenyl]-6-(4-methoxyphenyl)-1,3,5-triazine
2,4-bis-[{4-(2″-methylpropenyloxy)-2-hydroxy}-phenyl]-6-(4-methoxyphenyl)-1,3,5-triazine
2,4-bis-[{4-(1′,1′,1′,3′,5′,5′,5′-Heptamethylsiloxy-2″-methyl-propyloxy)-2-hydroxy}-phenyl]-6-(4-methoxyphenyl)-1,3,5-triazine
   UV-A filters, such as e.g.:
Terephthalylidene-dibornanesulfonic acid and salts (Mexoryl®SX)
4-t-Butyl-4′-methoxy-dibenzoylmethane (avobenzone)/(Neo Heliopan®357)
phenylene-bis-benzimidazyl-tetrasulfonic acid disodium salt (Neo Heliopan®AP)
2,2′-(1,4-phenylene)-bis-(1H-benzimidazole-4,6-disulfonic acid), monosodium salt
2-(4-diethylamino-2-hydroxybenzoyl)-benzoic acid hexyl ester (Uvinul® A Plus)
4-isopropyldibenzoylmethane
Indanylidene compounds according to DE 100 55 940 (=WO 02/38537)
   In this context, UV absorbers which are particularly suitable for combination are
p-aminobenzoic acid
3-(4′-trimethylammonium)-benzylidene-bornan-2-one methyl sulfate
salicylic acid homomethyl ester (Neo-Heliopan®HMS)
2-hydroxy-4-methoxy-benzophenone (Neo Heliopan®BB)
2-phenylbenzimidazolesulfonic acid (Neo Heliopan®Hydro)
terephthalylidene-dibornanesulfonic acid and salts (Mexoryl®SX)
4-tert-butyl-4′-methoxydibenzoylmethane (Neo Heliopan®357)
3-(4′-sulfo)benzylidene-bornan-2-one and salts
2-ethylhexyl 2-cyano-3,3-diphenylacrylate (Neo Heliopan®303)
N-[(2 and 4)-[2-(oxoborn-3-ylidene)methyl]benzyl]-acrylamide polymer
p-methoxycinnamic acid 2-ethyl hexyl ester (Neo Heliopan® AV)
p-aminobenzoic acid ethyl ester (25 mol) ethoxylated
p-methoxycinnamic acid isoamyl ester (Neo Heliopan®E 1000)
2,4,6-trianilino-(p-carbo-2′-ethylhexyl-1′-oxy)-1,3,5-triazine (Uvinal®T150)
phenol, 2-(2H-benzotriazol-2-yl)-4-methyl-6-(2-methyl-3-(1,3,3,3-tetramethyl-1-(trimethylsilyl)-oxy)-disiloxyanyl)-propyl), (Mexoryl®XL)
4,4′-[(6-[4-(1,1-dimethyl)-aminocarbonyl)-phenylamino]-1,3,5-triazine-2,4-diyl)-diimino]-bis-(benzoic acid 2-ethylhexyl ester), (UvasorbHEB)
3-(4′-methylbenzylidene)-d,l-camphor (Neo Heliopan®MBC)
3-benzylidenecamphor
salicylic acid 2-ethylhexyl ester (Neo-Heliopan®OS)
4-dimethylaminobenzoic acid 2-ethylhexyl ester (Padimate O)
hydroxy-4-methoxy-benzophenone-5-sulfonic acid and Na salt
2,2′-methylene-bis-(6-(2H-benzotriazol-2-yl)-4-1,1,3,3-tetramethylbutyl)-phenol), (Tinosorb®M)
phenylene-bis-benzimidazyl-tetrasulfonic acid disodium salt (Neo Heliopan®AP)
2,4-bis-[{(4-(2-ethyl-hexyloxy)-2-hydroxy}-phenyl]-6-(4-methoxyphenyl)-1,3,5-triazine, (Tinosorb®S)
benzylidenemalonate-polysiloxane (Parsol®SLX)
menthyl anthranilate (Neo Heliopan®MA)
2-(4-diethylamino-2-hydroxybenzoyl)-benzoic acid hexyl ester (Uvinul® A Plus)
indanylidene compounds according to DE 100 55 940 (=WO 02138537)

Particulate UV filters or inorganic pigments, which can optionally be hydrophobized, such as the oxides of titanium (TiO₂), zinc (ZnO), iron (Fe₂O₃), zirconium (ZrO₂), silicon (SiO₂), manganese (e.g. MnO), aluminium (Al₂O₃), cerium (e.g. Ce₂O₃) and/or mixtures, can furthermore be employed.

Preparations according to the invention including the preferred embodiments for constituents (a) and/or (b) optionally comprise further constituents having care properties, such as, for example, fatty alcohols having 6-30 C atoms. The fatty alcohols here can be saturated or unsaturated and linear or branched. Furthermore, these fatty alcohols can in some cases be a constituent of the oily phase (vii) if they correspond to the definition given there. Alcohols which can be employed are, for example, decanol, decenol, octanol, octenol, dodecanol, dodecenol, octadienol, decadienol, dodecadienol, oleyl alcohol, ricinoleyl alcohol, erucyl alcohol, stearyl alcohol, isostearyl alcohol, cetyl alcohol, lauryl alcohol, myristyl alcohol, arachidyl alcohol, caprylyl alcohol, capryl alcohol, linoleyl alcohol, linolenyl alcohol and behenyl alcohol, as well as Guerbet alcohols thereof, such as, for example, 2-octyl-1-dodecanol, it being possible for the list to be extended virtually as desired by further alcohols of related structural chemistry. The fatty alcohols preferably originate from natural fatty acids, being conventionally prepared from the corresponding esters of the fatty acids by reduction. Fatty alcohol fractions which are formed by reduction from naturally occurring fats and fatty oils, such as e.g. beef tallow, groundnut oil, colza oil, cottonseed oil, soya oil, sunflower oil, palm kernel oil, linseed oil, maize oil, castor oil, rape oil, sesame oil, cacao butter and coconut fat, can furthermore be employed.

Substances having care properties which can be employed in an outstanding manner in the preparations according to the invention comprising a mixture comprising or consisting of (a) one or more compounds of formula 1 and (b) one or more compounds selected from the group consisting of (i) chelating agents, (ii) phenolic derivatives, plant extracts with an amount of phenolic derivatives and (iii) organic acid derivatives including the preferred embodiments for constituents (a) and/or (b) moreover include
ceramides, where ceramides are understood as meaning N-acylsphingosins (fatty acid amides of sphingosin) or synthetic analogues of such lipids (so-called pseudo-ceramides), which significantly improve the water retention capacity of the stratum corneum.
phospholipids, for example soya lecithin, egg lecithin and cephalins
fatty acids
phytosterols and phytosterol-containing fats or waxes
vaseline, paraffin oils and silicone oils; the latter include, inter alia, dialkyl- and alkylarylsiloxanes, such as dimethylpolysiloxane and methylphenylpolysiloxane, as well as alkoxylated and quaternized derivatives thereof.

Animal and/or plant protein hydrolysates can advantageously also be added to the preparations according to the invention including the preferred embodiments for constituents (a) and/or (b). Substances which are advantageous in this respect are, in particular, elastin, collagen, keratin, milk protein, soya protein, oat protein, pea protein, almond protein and wheat protein fractions or corresponding protein hydrolysates, and also condensation products thereof with fatty acids and quaternized protein hydrolysates, the use of plant protein hydrolysates being preferred.

The preparations according to the invention which comprise a mixture comprising or consisting of (a) one or more compounds of formula 1 and (b) one or more compounds selected from the group consisting of (i) chelating agents, (ii) phenolic derivatives, plant extracts with an amount of phenolic derivatives and (iii) organic acid derivatives including the preferred embodiments for constituents (a) and/or (b) can also comprise antioxidants, it being possible for all the antioxidants which are suitable or usual for cosmetic and/or dermatological uses to be used. The antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. alpha-carotene, beta-carotene, lycopene) and derivatives thereof, liponic acid and derivatives thereof (e.g. dihydroliponic acid), aurothioglucose, propyl-thiouracil and other thiols (e.g. thioredoxin, glutathione, cysteine, cystine, cystamine and glycosyl, N-acetyl, methyl, ethyl, propyl, amyl, butyl and lauryl, palmitoyl, oleyl, gamma-linoleyl, cholesteryl and glyceryl esters thereof) as well as salts thereof dilauryl thiodipropionate, distearyl thiodipropionate, thiodipropionic acid and derivatives thereof (esters, ethers, peptides, lipids, nucleotides, nucleosides and salts) as well as sulfoximine compounds (e.g. buthionine sulfoximine, homocysteine sulfoximine, buthionine sulfones, penta-, hexa-, heptathionine sulfoximine) in very low tolerated dosages, furthermore (metal) chelators, e.g. alpha-hydroxy-fatty acids, palmitic acid, phytic acid, lactoferrin, alpha-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. gamma-linolenic acid, linoleic acid, oleic acid), folic acid and derivatives thereof, ubiquinone and ubiquinol and derivatives thereof, vitamin C derivatives (e.g. ascorbyl palmitate, Mg ascorbyl phosphate, ascorbyl acetate), tocopherols and derivatives thereof (e.g. vitamin E, vitamin E acetate), vitamin A and derivatives thereof (vitamin A palmitate) as well as coniferylbenzoate of benzoin resin, rutic acid and derivatives thereof, ferulic acid and derivatives thereof, butylhydroxytoluene, butylhydroxyanisole, nordihydroguaiac resin acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenium methionine), stilbenes and derivatives thereof (e.g. stilbene oxide, trans-stilbene oxide) and derivatives (salts, esters, ethers, sugars, nucleotides, nucleosides, peptides and lipids) of these active compounds mentioned.

The preparations according to the invention which comprise a mixture comprising or consisting of (a) one or more compounds of formula 1 and (b) one or more compounds selected from the group consisting of (i) chelating agents, (ii) phenolic derivatives, plant extracts with an amount of phenolic derivatives and (iii) organic acid derivatives including the preferred embodiments for constituents (a) and/or (b) can advantageously also comprise vitamins and vitamin precursors, it being possible for all the vitamins and vitamin precursors which are suitable or usual for cosmetic and/or dermatological uses to be used. There are worth mentioning here, in particular, vitamins and vitamin precursors, such as tocopherols, vitamin A, niacin acid and niacinamide, further vitamins of the B complex, in particular biotin, and vitamin C and panthenol and derivatives thereof, in particular the esters and ethers of panthenol and cationically derivatized panthenols, such as e.g. panthenol triacetate, panthenol monoethyl ether and the monoacetate thereof and cationic panthenol derivatives.

The preparations according to the invention, which advantageously comprise a mixture comprising or consisting of (a) one or more compounds of formula 1 and (b) one or more compounds selected from the group consisting of (i) chelating agents, (ii) phenolic derivatives, plant extracts with an amount of phenolic derivatives and (iii) organic acid derivatives including the preferred embodiments for constituents (a) and/or (b), can also comprise antiinflammatory and/or redness- and/or itching-alleviating active compounds. All the antiinflammatory or redness- and/or itching-alleviating active compounds which are suitable or usual for cosmetic and/or pharmaceutical, in particular dermatological uses can be used here. Antiinflammatory and redness- and/or itching-alleviating active compounds which are advantageously employed are steroidal antiinflammatory substances of the corticosteroid type, such as e.g. hydrocortisone, dexamethasone, dexamethasone phosphate, methylprednisolone or cortisone, it being possible for the list to be extended by addition of further steroidal antiinflammatories. Non-steroidal antiinflammatories can also be employed. There are to be mentioned here by way of example oxicams, such as piroxicam or tenoxicam; salicylates, such as aspirin, Disalcid, Solprin or fendosal; acetic acid derivatives, such as diclofenac, fenclofenac, indomethacin, sulindac, tolmetin or clindanac; fenamates, such as mefenamic, meclofenamic, flufenamic or niflumic; propionic acid derivatives, such as ibuprofen, naproxen, benoxaprofen or pyrazoles, such as phenylbutazone, oxyphenylbutazone, febrazone or azapropazone. Alternatively, natural antiinflammatory or redness- and/or itching-alleviating substances can be employed. Plant extracts, specific highly active plant extract fractions and highly pure active substances isolated from plant extracts can be employed. Extracts, fractions and active substances from camomile, aloe vera, Commiphora species, Rubia species, willow, rose-bay willow herb, oats as well as pure substances, such as, inter alia, bisabolol, apigenin 7-glucoside, boswellic acid, phytosterols, glycyrrhizic acid, glabridin or licochalcone A, are particularly preferred. The preparations comprising diphenylmethane derivatives of the formula 1 can also comprise mixtures of two or more antiinflammatory active compounds.

Bisabolol, boswellic acid, as well as extracts and isolated highly pure active compounds from oats and Echinacea are particularly preferred for use in the context of the invention, and alpha-bisabolol and extracts and isolated highly pure active compounds from oats are especially preferred.

The amount of antiirritants (one or more compounds) in the preparations is preferably 0.0001 to 20 wt. %, particularly preferably 0.0001 to 10 wt. %, in particular 0.001 to 5 wt. %, based on the total weight of the preparation.

The preparations according to the invention which comprise a mixture comprising or consisting of (a) one or more compounds of formula 1 and (b) one or more compounds selected from the group consisting of (i) chelating agents, (ii) phenolic derivatives, plant extracts with an amount of phenolic derivatives and (iii) organic acid derivatives including the preferred embodiments for constituents (a) and/or (b) can advantageously also comprise moisture retention regulators. The following substances e.g. are used as moisture retention regulators (moisturizers): sodium lactate, urea, alcohols, sorbitol, glycerol, propylene glycol, collagen, elastin or hyaluronic acid, diacyl adipates, petrolatum, ectoin, urocanic acid, lecithin, pantheol, phytantriol, lycopene, algae extract, ceramides, cholesterol, glycolipids, chitosan, chondroitin sulfate, polyamino acids lanolin, lanolin esters, amino acids, alpha-hydroxy acids (e.g. citric acid, lactic acid, malic acid) and derivatives thereof, sugars (e.g. inositol), alpha-hydroxy-fatty acids, phytosterols, triterpene acids, such as betulinic acid or ursolic acid, algae extracts.

The preparations according to the invention which comprise a mixture comprising or consisting of (a) one or more compounds of formula 1 and (b) one or more compounds selected from the group consisting of (i) chelating agents, (ii) phenolic derivatives, plant extracts with an amount of phenolic derivatives and (iii) organic acid derivatives including the preferred embodiments for constituents (a) and/or (b) can advantageously also comprise mono-, di- and oligosaccharides, such as, for example, glucose, galactose, fructose, mannose, laevulose and lactose.

The preparations according to the invention which comprise a mixture comprising or consisting of (a) one or more compounds of formula 1 and (b) one or more compounds selected from the group consisting of (i) chelating agents, (ii) phenolic derivatives, plant extracts with an amount of phenolic derivatives and (iii) organic acid derivatives including the preferred embodiments for constituents (a) and/or (b) can advantageously also comprise plant extracts, which are conventionally prepared by extraction of the whole plant, but also in individual cases exclusively from blossom and/or leaves, wood, bark or roots of the plant. In respect of the plant extracts which can be used, reference is made in particular to the extracts which are listed in the table starting on page 44 of the 3rd edition of the Leitfaden zur Inhaltsstoffdeklaration kosmetischer Mittel [Manual of Declaration of the Constituents of Cosmetic Compositions], published by Industrieverband Korperpflegemittel und Waschmittel e.V. (IKW), Frankfurt. Extracts which are advantageous in particular are those from aloe, witch hazel, algae, oak bark, rose-bay willow-herb, stinging nettle, dead nettle, hops, camomile, yarrow, arnica, calendula, burdock root, horsetail, hawthorn, linden blossom, almond, pine needle, horse chestnut, sandalwood, juniper, coconut, mango, apricot, orange, lemon, lime, grapefruit, apple, green tea, grapefruit pip, wheat, oats, barley, sage, thyme, wild thyme, rosemary, birch, mallow, lady's smock, willow bark, restharrow, coltsfoot, hibiscus, ginseng and ginger root. In this context, the extracts from aloe vera, camomile, algae, rosemary, calendula, ginseng, cucumber, sage, stinging nettle, linden blossom, arnica and witch hazel are particularly preferred. Mixtures of two or more plant extracts can also be employed. Extraction agents which can be used for the preparation of the plant extracts mentioned are, inter alia, water, alcohols and mixtures thereof. In this context, among the alcohols lower alcohols, such as ethanol and isopropanol, but also polyhydric alcohols, such as ethylene glycol, propylene glycol and butylene glycol, are preferred, and in particular both as the sole extraction agent and in mixtures with water. The plant extracts can be employed both in the pure and in the diluted form.

Preparations according to the invention including the preferred embodiments for constituents (a) and/or (b) can in numerous cases advantageously comprise the following preservatives. Preservatives which are preferably chosen here are those such as benzoic acid, its esters and salts, propionic acid and its salts, salicylic acid and its salts, 2,4-hexadienoic acid (sorbic acid) and its salts, formaldehyde and paraformaldehyde, 2-hydroxybiphenyl ether and its salts, 2-zinc-sulfidopyridine N-oxide, inorganic sulfites and bisulfites, sodium iodate, chlorobutanolum, 4-ethylmercury(II)-5-amino-1,3-bis(2-hydroxybenzoic acid), its salts and esters, dehydracetic acid, formic acid, 1,6-bis(4-amidino-2-bromophenoxy)-n-hexane and its salts, the sodium salt of ethylmercury(II)-thiosalicylic acid, phenylmercury and its salts, 10-undecylenic acid and its salts, 5-amino-1,3-bis(2-ethylhexyl)-5-methyl-hexahydropyrimidine, 5-bromo-5-nitro-1,3-dioxane, 2-bromo-2-nitro-1,3-propanediol, 2,4-dichlorobenzyl alcohol, N-(4-chlorophenyl)-N′-(3,4-dichlorophenyl)-urea, 4-chloro-m-cresol, 2,4,4′-trichloro-2′-hydroxy-diphenyl ether, 4-chloro-3,5-dimethylphenol, 1,1′-methylene-bis(3-(1-hydroxymethyl-2,4-dioximidazolidin-5-yl)urea), poly-(hexamethylene diguanide) hydrochloride, 2-phenoxyethanol, hexamethylenetetramine, 1-(3-chloroallyl)-3,5,7-triaza-1-azonia-adamantane chloride, 1-(4-chlorophenoxy)-1-(1H-imidazol-1-yl)-3,3-dimethyl-2-butanone, 1,3-bis-(hydroxy-methyl)-5,5-dimethyl-2,4-imidazolidinedione, benzyl alcohol, Octopirox, 1,2-dibromo-2,4-dicyanobutane, 2,2′-methylene-bis(6-bromo-4-chloro-phenol), bromochlorophene, mixture of 5-chloro-2-methyl-3(2H)-isothiazolinone and 2-methyl-3(2H)-isothiazolinone with magnesium chloride and magnesium nitrate, 2-benzyl-4-chlorophenol, 2-chloroacetamide, chlorhexidine, chlorhexidine acetate, chlorhexidine gluconate, chlorhexidine hydrochloride, 1-phenoxy-propan-2-ol, N-alkyl(C₁₂-C₂₂)trimethyl-ammonium bromide and chloride, 4,4-dimethyl-1,3-oxazolidine, N-hydroxymethyl-N-(1,3-di(hydroxymethyl)-2,5-dioxoimidazolidin-4-yl)-N′-hydroxy-methylurea, 1,6-bis(4-amidino-phenoxy)-n-hexane and its salts, glutaraldehyde, 5-ethyl-1-aza-3,7-dioxabicyclo(3.3.0)octane, 3-(4-chlorophenoxy)-1,2-propanediol, hyamines, alkyl-(C₈-C₁₈)-dimethyl-benzyl-ammonium chloride, alkyl-(C₈-C₁₈)-dimethyl-benzylammonium bromide, alkyl-(C₈-C₁₈)-dimethyl-benzyl-ammonium saccharinate, benzyl hemiformal, 3-iodo-2-propynyl butylcarbamate or sodium hydroxymethyl-aminoacetate.

In various cases it may also be advantageous also to employ substances which are chiefly employed for inhibition of the growth of undesirable microorganisms on or in animal organisms in the preparations according to the invention comprising a mixture comprising or consisting of (a) one or more compounds of formula 1 and (b) one or more compounds selected from the group consisting of (i) chelating agents, (ii) phenolic derivatives, plant extracts with an amount of phenolic derivatives and (iii) organic acid derivatives, including the preferred embodiments for constituents (a) and/or (b). In this respect, in addition to conventional preservatives, further active compounds which are worth mentioning, in addition to the large group of conventional antibiotics, are, in particular, the products relevant for cosmetics, such as triclosan, climbazole, octoxyglycerol, Octopirox (1-hydroxy-4-methyl-6-(2,4,4-trimethylpentyl)-2(1H)-pyridone, 2-aminoethanol), chitosan, farnesol, glycerol monolaurate or combinations of the substances mentioned, which are employed, inter alia, against underarm odour, foot odour or dandruff formation.

Substances having a perspiration-inhibiting activity (antiperspirants) can moreover be particularly advantageously employed in the preparations according to the invention comprising a mixture comprising or consisting of (a) one or more compounds of formula 1 and (b) one or more compounds selected from the group consisting of (i) chelating agents, (ii) phenolic derivatives, plant extracts with an amount of phenolic derivatives and (iii) organic acid derivatives including the preferred embodiments for constituents (a) and/or (b), for combating body odour. Perspiration-inhibiting active compounds which are employed are, above all, aluminium salts, such as aluminium chloride, aluminium hydrochloride, nitrate, sulfate, acetate etc. In addition, however, the use of compounds of zinc, magnesium and zirconium may also be advantageous. For use in cosmetic and dermatological antiperspirants, the aluminium salts and—to a somewhat lesser extent—aluminium/zirconium salt combinations have substantially proved suitable. The aluminium hydroxychlorides which are partly neutralized and therefore tolerated better by the skin, but are not quite so active, are additionally worth mentioning. Alongside aluminium salts, further substances are also possible, such as, for example, a) protein-precipitating substances, such as, inter alia, formaldehyde, glutaraldehyde, natural and synthetic tannins and trichloroacetic acid, which bring about surface blockage of the sweat glands, b) local anaesthetics (inter alia dilute solutions of e.g. lidocaine, prilocalne or mixtures of such substances), which eliminate sympathetic supply of the sweat glands by blockade of the peripheral nerve pathways, c) zeolites of the X, A or Y type, which, alongside the reduction in secretion of perspiration, also function as adsorbents for bad odours, and d) botulinus toxin (toxin of the bacterium Clostridium botulnum), which is also employed in cases of hyperhidrosis, a pathologically increased secretion of perspiration, and the action of which is based on an irreversible blocking of the release of the transmitter substance acetylcholine, which is relevant for secretion of perspiration.

Further to this the preparations according to the invention comprising a mixture comprising or consisting of (a) one or more compounds of formula 1 and (b) one or more compounds selected from the group consisting of (i) chelating agents, (ii) phenolic derivatives, plant extracts with an amount of phenolic derivatives and (iii) organic acid derivatives including the preferred embodiments for constituents (a) and/or (b) can further comprise one or more cooling agents. Individual cooling active compounds which are preferred for use in the context of the present invention are listed below. The person skilled in the art can supplement the following list with a large number of further cooling active compounds; the cooling active compounds listed can also be employed in combination with one another: l-menthol, d-menthol, racemic menthol, menthone glycerol acetal (trade name: Frescolat®MGA), menthyl lactate (trade name: Frescolat®ML, menthyl lactate is preferably l-menthyl lactate, in particular l-menthyl l-lactate), substituted menthyl-3-carboxylic acid amides (e.g. menthyl-3-carboxylic acid N-ethylamide), 2-isopropyl-N-2,3-trimethylbutanamide, substituted cyclohexanecarboxylic acid amides, 3-menthoxypropane-1,2-diol, 2-hydroxyethyl menthyl carbonate, 2-hydroxypropyl menthyl carbonate, N-acetylglycine menthyl ester, isopulegol, menthyl hydroxycarboxylic acid esters (e.g. menthyl 3-hydroxybutyrate), monomenthyl succinate, 2-mercaptocyclodecanone, menthyl 2-pyrrolidin-5-onecarboxylate, 2,3-dihydroxy-p-menthane, 3,3,5-trimethylcyclohexanone glycerol ketal, 3-menthyl 3,6-di- and -trioxaalkanoates, 3-menthyl methoxyacetate, icilin.

Preferred cooling active compounds are: l-menthol, d-menthol, racemic menthol, menthone glycerol acetal (trade name: Frescolat®MGA), menthyl lactate (preferably l-menthyl lactate, in particular l-menthyl l-lactate, trade name: Frescolat®ML), substituted menthyl-3-carboxylic acid amides (e.g. menthyl-3-carboxylic acid N-ethylamide), 2-isopropyl-N-2,3-trimethylbutanamide, substituted cyclohexanecarboxylic acid amides, 3-menthoxypropane-1,2-diol, 2-hydroxyethyl menthyl carbonate, 2-hydroxypropyl menthyl carbonate, isopulegol.

Particularly preferred cooling active compounds are: l-menthol, racemic menthol, menthone glycerol acetal (trade name: Frescolat®MGA), menthyl lactate (preferably 1-menthyl lactate, in particular l-menthyl l-lactate, trade name: Frescolat®ML), 3-menthoxypropane-1,2-diol, 2-hydroxyethyl menthyl carbonate, 2-hydroxypropyl menthyl carbonate.

Very particularly preferred cooling active compounds are: l-menthol, menthone glycerol acetal (trade name: Frescolat®MGA), menthyl lactate (preferably l-menthyl lactate, in particular 1-menthyl l-lactate, trade name: Frescolat®ML).

The use concentration of the cooling active compounds to be employed is, depending on the substance, preferably in the concentration range of from 0.01 to 20 wt. % and preferably in the concentration range of from 0.1 to 5 wt. %, based on the total weight of the finished (ready-to-use) cosmetic or pharmaceutical preparation.

The preparations according to the invention which comprise a mixture comprising or consisting of (a) one or more compounds of formula 1 and (b) one or more compounds selected from the group consisting of (i) chelating agents, (ii) phenolic derivatives, plant extracts with an amount of phenolic derivatives and (iii) organic acid derivatives including the preferred embodiments for constituents (a) and/or (b) can also comprise anionic, cationic, nonionic and/or amphoteric surfactants, especially if crystalline or microcrystalline solids, for example inorganic micropigments, are to be incorporated into the preparations. Surfactants are amphiphilic substances which can dissolve organic, nonpolar substances in water. According to the invention, surfactants therefore do not belong to the oily phase. In this context, the hydrophilic contents of a surfactant molecule are usually polar functional groups, for example —COO⁻, —OSO₃²⁻, —SO₃⁻, while the hydrophobic parts as a rule are nonpolar hydrocarbon radicals. Surfactants are in general classified according to the nature and charge of the hydrophilic molecular moiety. A distinction can be made between four groups here.
anionic surfactants,
cationic surfactants,
amphoteric surfactants and
nonionic surfactants.

Anionic surfactants as a rule contain carboxylate, sulfate or sulfonate groups as functional groups. In aqueous solution, they form negatively charged organic ions in an acid or neutral medium. Cationic surfactants are almost exclusively characterized by the presence of a quaternary ammonium group. In aqueous solution, they form positively charged organic ions in an acid or neutral medium. Amphoteric surfactants contain both anionic and cationic groups and accordingly behave like anionic or cationic surfactants in aqueous solution, depending on the pH. In a strongly acid medium they have a positive charge, and in an alkaline medium a negative charge. On the other hand, they are zwitter-ionic in the neutral pH range. Polyether chains are typical of nonionic surfactants. Nonionic surfactants do not form ions in an aqueous medium.

### A. Anionic Surfactants

Anionic surfactants which are advantageously to be used are acylamino acids (and salts thereof, such as
acyl glutamates, for example sodium acyl glutamate, di-TEA-palmitoyl aspartate and sodium caprylic/capric glutamate,
acyl peptides, for example palmitoyl hydrolysed milk protein, sodium cocoyl hydrolysed soya protein and sodium/potassium cocoyl hydrolysed collagen,
sarcosinates, for example myristoyl sarcosine, TEA-lauroyl sarcosinate, sodium lauroyl sarcosinate and sodium cocoyl sarcosinate,
taurates, for example sodium lauroyl taurate and sodium methylcocoyl taurate,
acyl lactylates, lauroyl lactylate, caproyl lactylate
alaninates
   carboxylic acids and derivatives, such as
for example, lauric acid, aluminium stearate, magnesium alkanolate and zinc undecylenate,
ester-carboxylic acids, for example calcium stearoyl lactylate, laureth-6 citrate and sodium PEG-4 lauramide carboxylate,
ether-carboxylic acids, for example sodium laureth-13 carboxylate and sodium PEG-6 cocamide carboxylate,
   phosphoric acid esters and salts, such as, for example, DEA-oleth-10 phosphate and dilaureth-4 phosphate,
   sulfonic acids and salts, such as
acyl isethionates, e.g. sodium/ammonium cocoyl isethionate,
alkylarylsulfonates,
alkylsulfonates, for example sodium coco-monoglyceride sulfate, sodium C₁₂₋₁₄ olefin-sulfonate, sodium lauryl sulfoacetate and magnesium PEG-3 cocamide sulfate,
sulfosuccinates, for example dioctyl sodium sulfosuccinate, disodium laureth-sulfosuccinate, disodium laurylsulfosuccinate and disodium undecylenamido-MEA-sulfosuccinate and
   sulfuric acid esters, such as
alkyl ether-sulfate, for example sodium, ammonium, magnesium, MIPA, TIPA laureth sulfate, sodium myreth sulfate and sodium C12-13 pareth sulfate,
alkyl sulfates, for example sodium, ammonium and TEA lauryl sulfate.

### B. Cationic Surfactants

Cationic surfactants which are advantageously to be used are
alkylamines,
alkylimidazoles,
ethoxylated amines and
quaternary surfactants,
   RNH₂CH₂CH₂COO⁻ (at pH=7)
   RNHCH₂CH₂COO— B⁺ (at pH=12) B⁺=any desired cation, e.g. Na⁺
ester quats

Quaternary surfactants contain at least one N atom which is covalently bonded to 4 alkyl or aryl groups. This leads to a positive charge, independently of the pH. Alkylbetaine, alkylamidopropylbetaine and alkylamidopropylhydroxysulfaine are advantageous. The cationic surfactants used can furthermore preferably be chosen from the group consisting of quaternary ammonium compounds, in particular benzyltrialkyl-ammonium chlorides or bromides, such as, for example, benzyldimethylstearyl-ammonium chloride, furthermore alkyltrialkylammonium salts, for example cetyltrimethylammonium chloride or bromide, alkyldimethylhydroxy-ethylammonium chlorides or bromides, dialkyldimethylammonium chlorides or bromides, alkylamide-ethyltrimethyl-ammonium ether-sulfates, alkylpyridinium salts, for example lauryl- or cetylpyrimidinium chloride, imidazoline derivatives and compounds having a cationic character, such as amine oxides, for example alkyldimethylamine oxides or alkylaminoethyldimethylamine oxides. Cetyltrimethyl-ammonium salts in particular are advantageously to be used.

### C. Amphoteric Surfactants

Amphoteric surfactants which are advantageously to be used are
a) acyl-/dialkylethylenediamine, for example sodium acylamphoacetato, disodium acylamphodipropionate, disodium alkylamphodiacetate, sodium acyl-amphohydroxy-propylsulfonate, disodium acylamphodiacetate and sodium acylamphopropionate,
b) N-alkylamino acids, for example aminopropyl alkylglutamide, alkylaminopropionic acid, sodium alkylimidodipropionate and lauroamphocarboxyglycinate.

### D. Nonionic Surfactants

Nonionic surfactants which are advantageously to be used are
alcohols,
alkanolamides, such as cocamides MEA/DEA/MIPA,
amine oxides, such as cocoamidopropylamine oxide,
esters which are formed by esterification of carboxylic acids with ethylene oxide, glycerol, sorbitan or other alcohols,
ethers, for example ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated/propoxylated lanolin, ethoxylated/propoxylated polysiloxanes, propoxylated POE ethers and alkyl polyglycosides, such as lauryl glucoside, decyl glycoside and coco-glycoside.
sucrose esters, sucrose ethers
polyglycerol esters, diglycerol esters, monoglycerol esters
methylglucose esters, esters of hydroxy acids

The use of a combination of anionic and/or amphoteric surfactants with one or more nonionic surfactants is furthermore advantageous.

In this context, the surface-active substance(s) can be present in a preparation according to the invention in an amount in the range of from 0.5 to 98 wt. %, based on the total weight of the preparation.

Preferred embodiments and further aspects of the present invention emerge from the attached patent claims and the following examples. Unless stated otherwise, all the data relate to the weight.

### EXAMPLE 1

In vitro experiments on the tyrosinase-inhibiting activity of preparations according to the invention comprising styrylresorcinol of formula 3 (CARN: 85-27-8; 4(1-phenylethyl)-1,3-dihydroxybenzen) and one or more compounds selected from the group consisting of (i) chelating agents, (ii) phenolic derivatives, plant extracts with an amount of phenolic derivatives and (iii) organic acid derivatives.

The finding that mixtures of styrylresorcinol of formula 3 together with one or more compounds selected from the group consisting of (i) chelating agents, (ii) phenolic derivatives, plant extracts with an amount of phenolic derivatives and (iii) organic acid derivatives improve the skin and/or hair lightening and/or senile keratosis reducing activity in a synergistic way is based on experiments on the inhibition on the enzyme tyrosinase.

### Principle of the Tyrosinase-Assay:

Tyrosinase, the speed determining key enzyme in the melanin-biosynthesis, catalyses the oxidation of L-tyrosine and L-3,4-dihydroxyphenylalanine (L-DOPA) to dopachrome. The latter is oxidised to melanin catalysed by various enzymes via several steps. The principle of the assay is to oxidise L-DOPA to dopachrome with or without the test substance under physiological conditions in a controlled reaction. Dopachrome and dopaquinone is detected via photometric detection methods. Commercially available fungal-tyrosinase is used for testing the enzyme activity and the correlating inhibition of melanin production.

As testing material styrylresorcinol of formula 3 (more than 99% purity) as constituent (a), specific compounds of the constituent b) selected from the group consisting of (i) chelating agents, (ii) phenolic derivatives, plant extracts with an amount of phenolic derivatives and (iii) organic acid derivatives as well as mixtures thereof are used.

The enzymatic reaction was conducted with or without a potential inhibitor of the tyrosinase of constituents (a) and (b), such as styrylresorcinol of formula 3 (more than 99% purity) of constituent (a) alone, specific compounds of the constituent b) selected from the group consisting of (i) chelating agents, (ii) phenolic derivatives, plant extracts with an amount of phenolic derivatives and (iii) organic acid derivatives alone, as well as mixtures thereof.

The concentrations of the inhibitor or a mixture thereof range from 0.1 to 1000 μg/ml. The enzyme activity was adjusted to 50 u/ml fungal-tyrosinase in 66.7 mM phosphate buffer (pH=6.8). The reaction mixture was incubated for 10 minutes in a 96-well microtiter plate in a horizontal agitator at 37° C. Then L-DOPA (end concentration: 0.1 mg/ml). The enzymatic conversion to dopachrome is related to the inhibition activity of the single test substances or the mixture thereof, which is shown in a brown discolouration, which can be detected at 475 nm. In one 96-well microtiter plate 3 testing compounds each in 5 concentrations were tested. For each concentration level 3 tests were conducted in two independent experiments. As a standard kojic acid (BIO 165) was used.

### Results:

The experiments showed that the mixtures of styrylresorcinol of formula 3 together with one or more compounds of the constituent (b) selected from the group consisting of (i) chelating agents, (ii) phenolic derivatives, plant extracts with an amount of phenolic derivatives and (iii) organic acid derivatives in the specific weight ratios listed in table 2 had an synergistic effect on the inhibition on the enzyme tyrosinase. The improvement on the synergistic activity could also be calculated with the Kulls' equation (F. C. Kull et al., Applied Microbiology 9, 538-541 (1961); D. C. Steinberg, Cosmetics & Toiletries 115 (11), 59 (2000)).

TABLE 2
Ratios of 4-(1-phenylthyl)-1,3-dihydroxybenzene (CARN: 85-27-8) and
another lightening agent selected from groups (i), (ii) and (iii) showing
synergistic enhanced lightening activity in the tyrosinase assay.

| (i) Chelating | Ratio 4-(1-phenylthyl)-1,3-dihydroxybenzene | | | | | |
|---|---|---|---|---|---|---|
| Agents | CARN 85-27-8:(i) Chelating Agent | | | | | |
| Kojic acid | 1:20 | 1:8 | 1.3 | 1:2 | 1:1 | 2:1 |
| EDTA | 1:2 | 1:1 | 5:1 | 15:1 | 10:1 | 1:50 |
| Hinokitiol | 1:5 | 1:4 | 1:1 | 3:1 | 5:1 | 10:1 |
| Tropolone | 1:5 | 1:3 | 1:1 | 3:1 | 10:1 | 20:1 |
| Ascorbic acid | 1:20 | 1:20 | 1:3 | 1:2 | 1:1 | 2:1 |
| Lactic acid | 1:20 | 1:20 | 1:2 | 1:1 | 2:1 | 5:1 |
| Salicylic acid | 1:20 | 1:20 | 1:2 | 1:1 | 2:1 | 5:1 |
| Glycolic acid | 1:20 | 1:20 | 1:2 | 1:1 | 2:1 | 5:1 |
| Citric acid | 1:20 | 1:20 | 1:2 | 1:1 | 2:1 | 5:1 |
| Malic acid | 1:20 | 1:20 | 1:2 | 1:1 | 2:1 | 5:1 |
| (ii) Phenolic | | | | | | |
| compounds | | | | | | |
| and plant | | | | | | |
| extracts | | | | | | |
| comprising | Ratio 4-(1-phenylthyl)-1,3-dihydroxybenzene | | | | | |
| phenolic | CARN 85-27-8:(ii) Phenolic derivatives and | | | | | |
| compounds | plant extracts comprising phenolic derivatives | | | | | |
| Arbutine | 1:20 | 1:20 | 1:3 | 1:2 | 1:1 | 2:1 |
| Hydroquinone | 1:20 | 1:20 | 1:2 | 1:1 | 2:1 | 5:1 |
| Resorcinol | 1:20 | 1:8 | 1:3 | 1:2 | 1:1 | 2:1 |
| 4- | 1:20 | 1:8 | 1:3 | 1:2 | 1:1 | 2:1 |
| Butylresorcinol | | | | | | |
| Bearberry | 1:20 | 1:20 | 1:3 | 1:2 | 1:1 | 2:1 |
| extract | | | | | | |
| Pinus extract | 1:20 | 1:20 | 1:3 | 1:2 | 1:1 | 2:1 |
| Mulberry | 1:20 | 1:20 | 1:3 | 1:2 | 1:1 | 2:1 |
| extract | | | | | | |
| Soybean | 1:20 | 1:20 | 1:3 | 1:2 | 1:1 | 2:1 |
| extract | | | | | | |
| Artocarpus | 1:20 | 1:20 | 1:3 | 1:2 | 1:1 | 2:1 |
| extract | | | | | | |
| Licorice extract | 1:20 | 1:20 | 1:3 | 1:2 | 1:1 | 2:1 |
| (iii) Organic | Ratio 4-(1-phenylthyl)-1,3- | | | | | |
| acid | dihydroxybenzene CARN 85-27-8:(iii) | | | | | |
| derivatives | Organic acid derivatives | | | | | |
| Azelaic acid | 1:20 | 1:20 | 1:3 | 1:2 | 1:1 | 2:1 |
| 9- | 1:20 | 1:20 | 1:3 | 1:2 | 1:1 | 2:1 |
| Octadecenedioic | | | | | | |
| acid | | | | | | |
| alpha-Lipoic | 1:5 | 1:3 | 1:2 | 1:1 | 2:1 | 5.1 |
| acid | | | | | | |
| Retinoic acid | 1:2 | 5:1 | 5:1 | 15:1 | 50:1 | 20:1 |
| Niacinamide | 1:20 | 1:8 | 1:3 | 1:2 | 1:1 | 2:1 |
| Undecylenoyl | 1:20 | 1:8 | 1:3 | 1:2 | 1:1 | 2:1 |
| Phenylalanine | | | | | | |

### EXAMPLE 2

Examples of preparations according the invention comprising synergistic active mixtures of one or more diphenylmethane derivatives of formula 1 as constituent (a) and one or more skin and/or hair lightening and/or senile keratosis reducing compounds selected from the group consisting of (i) chelating agents, (ii) phenolic derivatives, plant extracts with an amount of phenolic derivatives and (iii) organic acid derivatives as constituent (b).

Cosmetic and pharmaceutical preparations according to the invention which show particularly enhanced synergistic effects on skin and/or hair lightening and/or senile keratosis reduction are further described in tables 3, 4 and 5. A further improvement on the skin and/or hair lightening and/or senile keratosis reducing activity is effected when the combination is further combined with one or more skin and/or hair lightening and/or senile keratosis reducing agents. Preferred embodiments of the present invention emerge from the following examples and the attached patent claims.

TABLE 3
Cosmetic and pharmaceutical, in particular dermatological preparations
comprising styrylresorcinol of formula 3 (CARN: 85-27-8; 4-(1-phenylethyl)-1,3-
dihydroxybenzene) and one or more skin and/or hair lightening and/or senile
reducing compounds of constituent b) selected from the group (i) chelating agents.

| RAW MATERIAL NAME | | | | | | | | | | | |
|---|---|---|---|---|---|---|---|---|---|---|---|
| (MANUFACTURER) | INCI | 1 | 2 | 3 | 4 | 5 | 6 | 7 | 8 | 9 | 10 |
| Skin lightener | | | | | | | | | | | |
| 4-(1-Phenylethyl)- | Phenylethyl Resorcinol | 0.10 | 0.25 | 0.50 | 0.75 | 1.00 | 2.00 | 0.10 | 0.50 | 1.00 | 0.5 |
| 1,3- | | | | | | | | | | | |
| dihydroxybenzene | | | | | | | | | | | |
| (CARN: 85-278) | | | | | | | | | | | |
| Kojic acid | Kojic acid | | | 1.50 | | | | | | 1.00 | |
| EDTA | EDTA | | | | 0.05 | | | 0.10 | | | 0.05 |
| Isopropyltropolone | Hinokitiol | | | | | | 0.20 | | 0.1 | | |
| Tropolone | Tropolone | | | | | 0.10 | | | | | |
| Ascorbic acid | Ascorbic acid | | | 1.50 | | | | | | | |
| Lactic acid | Lactic acid | | 5.00 | | | | | 2.00 | | | 1.50 |
| Salicylic acid | Salicylic acid | | | | | 0.50 | | | | | 1.50 |
| Glycolic acid | Glycolic acid | | | 1.00 | | | | | 1.50 | | |
| Citric acid | Citric acid | | 5.00 | | | | | | | | |
| Malic acid | Malic acid | 2.00 | | | | | | | | 0.50 | |
| Oil components | | | | | | | | | | | |
| Abil 100 ® | Dimethicone | 1.0 | 0.3 | | | | 1.0 | | | 2.0 | 0.3 |
| (Goldschmidt) | | | | | | | | | | | |
| Ascorbyl-6 Palmitate | Ascorbyl Palmitate | 0.1 | | 0.2 | | | | | 0.1 | | |
| Cetiol OE (Cognis) | Dicaprylyl Ether | | | | | | 3.0 | | | | |
| Corapan TQ ® | Diethylhexyl-2,6- | | 2.0 | | | | | | | | |
| (Symrise) | Naphtalate | | | | | | | | | | |
| Dragoxat EH | Ethylhexyl | | | 1.0 | 1.0 | | | | | | |
| (Symrise) | Ethylhexanoate | | | | | | | | | | |
| Isoadipate (Symrise) | Diisopropyl Adipate | | | | 1.0 | | | | | | |
| Isopropylmyristat | Isopropyl Myristate | | | | | | | | | | 4.0 |
| (Symrise) | | | | | | | | | | | |
| Isodragol (Symrise) | Triisononanoin | | | | | | | | | 6.0 | |
| Neutralöl (Symrise) | Caprylic/Capric/ | 4.0 | 2.0 | | | 2.0 | | | | | |
| | Triglyceride | | | | | | | | | | |
| Paraffin Oil | Mineral Oil | | | | | | | | | | 4.0 |
| PCL Liquid 100 | Cetearyl Octanoate | 4.0 | 3.0 | | | | | | 3.0 | | |
| (Symrise) | | | | | | | | | | | |
| Tegosoft TN ® | C12-C15 Alkylbenzoate | | | | 4.0 | 2.0 | | | | | |
| (Goldschmidt) | | | | | | | | | | | |
| Further ingredients | | | | | | | | | | | |
| Aloe Vera Gel | Water (Aqua). | 0.05 | 0.1 | | | | | | | | 0.1 |
| Konzentrat 10/1 | Aloe Barbadensis | | | | | | | | | | |
| (Symrise) | Leaf Extract | | | | | | | | | | |
| Arlypon F | Laureth-2 | | | | | | | 2.0 | | | |
| Ascorbyl-6 Palmitate | Ascorbyl Palmitate | 0.1 | | 0.2 | | | | | 0.1 | | |
| alpha-Bisabolol | Bisabolol | | 0.1 | | 0.2 | 0.1 | 0.1 | 0.1 | | | |
| (Symrise) | | | | | | | | | | | |
| 1,3-Butylenglycol | 1,3-Butylenglycol | | 3.0 | | | | | | | | |
| Carbopol 2050 ® | Carbomer | | 0.2 | | 0.5 | 0.1 | | | | | |
| (B. F. Goodrich) | | | | | | | | | | | |
| Ceramid Bio 391 | Ceramid | 0.3 | | | | | | | | | 0.1 |
| (Symrise) | | | | | | | | | | | |
| Citric Acid | Citric Acid | | | | | | | | 0.1 | 0.3 | |
| Copherol 1250 ® | Tocopherol acetate | | 0.5 | | 0.5 | 0.5 | 0.5 | | | | |
| (Cognis) | | | | | | | | | | | |
| Crinipan ® AD | Climbazol | | | | | | | 0.5 | | | |
| (Symrise) | | | | | | | | | | | |
| Dehyquart SP | Quaternium-52 | | | | | | | | 0.5 | | |
| Dehyton K | Cocamidopropyl Betaine | | | | | | | 12.0 | | | |
| Diethylenetriamine | Pentetic Acid | 0.05 | 0.1 | | | | | | | | 0.05 |
| Pentaacetic acid | | | | | | | | | | | |
| Dow Corning ® 193 | Dimethicon-Polyol | | | 1.0 | | | | | | | |
| (Dow corning) | | | | | | | | | | | |
| D-Panthenol (BASF) | Panthenol | | | 0.5 | | | 0.5 | 0.4 | | | |
| Dragocid Liquid | Phenoxyethanol (and) | 0.8 | | 0.8 | 0.8 | 0.8 | 0.8 | 0.5 | 0.8 | 0.8 | |
| (Symrise) | Methylparaben (and) | | | | | | | | | | |
| | Ethylparaben (and) | | | | | | | | | | |
| | Butylparaben (and) | | | | | | | | | | |
| | Propylparaben (and) | | | | | | | | | | |
| | Isobutylparaben | | | | | | | | | | |
| Dragophos S | Sodium Dihydroxycetyl | | | | | | | | | 2.0 | |
| (Symrise) | Phosphate | | | | | | | | | | |
| Dracorin CE | Glyceryl Stearate/Citrate | | | | | | | | | 1.0 | |
| Dracorin GMS | Glyceryl Stearate | 2.0 | 2.0 | | | 2.0 | | | | 2.0 | 3.0 |
| (Symrise) | | | | | | | | | | | |
| Dracorin 100 s.e. P | Glyceryl Stearate, PEG- | 3.0 | | | | | | | | | 8.0 |
| (Symrise) | 100 Stearate | | | | | | | | | | |
| Edeta BD ® (BASF) | Dinatrium-EDTA | 0.1 | 0.1 | | 0.1 | 0.1 | 0.1 | | | 0.05 | 0.05 |
| Emulgin B2 ® | Ceteareth-20 | 1.0 | | | | | | | 0.7 | | |
| (Cognis) | | | | | | | | | | | |
| Emulsiphos | Potassium | | 1.5 | | | 1.5 | | | | | |
| (Symrise) | Cetylphosphate, | | | | | | | | | | |
| | Hydrogenated Palm | | | | | | | | | | |
| | Glycerides | | | | | | | | | | |
| Ethylic alcohol | Ethyl alcohol | | | 13.0 | 5.0 | | | | | | |
| (96%) | | | | | | | | | | | |
| Extrapon Kamille | Water (Aqua), Propylene | | | 1.0 | | | | | | | |
| (Symrise) | Glycol, Butylene Glycol, | | | | | | | | | | |
| | Chamomilla Recitita | | | | | | | | | | |
| | (Matricaria) Flower | | | | | | | | | | |
| | Extract, Glucose, | | | | | | | | | | |
| | Bisabolol | | | | | | | | | | |
| Extrapon | Glycerin, Water (Aqua), | | | 1.0 | | | | | | | |
| Hamamelis | Hamamelis Virginiana | | | | | | | | | | |
| (Symrise) | (Witch Hazel) | | | | | | | | | | |
| | Bark/Leaves | | | | | | | | | | |
| Frescolat ML | Menthyl Lactate | | | | 0.5 | | | | | | |
| Glycerin 99% | Glycerin | 3.0 | | 4.5 | | 3.0 | 4.0 | | | | |
| Hydrolite-5 | Pentylene Glycol | | | | 4.5 | | 5.0 | | | 3.0 | |
| (Symrise) | | | | | | | | | | | |
| Keltrol T ® (Kelco) | Xanthan Gum | | 0.2 | 0.2 | 0.3 | | | | | | |
| Lanette E ® (Cognis) | Natriumcetearylsulfat | | 0.7 | | | | | | | | |
| Lanette O ® (Cognis) | Cetaeryl Alcohol | 1.1 | | | | | | | 2.5 | | |
| Lanette 16 ® | Cetylalkohol | | 1.2 | | | 0.5 | | | | | 2.0 |
| (Cognis) | | | | | | | | | | | |
| Lanette 18 (Care | Stearyl Alcohol | | | | | | | | | 4.5 | |
| Chemicals) | | | | | | | | | | | |
| Lara Care A-200 | Galactoarabinan | | | | | | 0.2 | | | | |
| (Rahn) | | | | | | | | | | | |
| Magnesium L- | Magnesium Ascorbyl | 0.1 | | 0.05 | | 0.3 | | | | | 0.1 |
| Ascorbyl-2- | Palmitate | | | | | | | | | | |
| phosphate | | | | | | | | | | | |
| NaOH 10% sol.. | Sodium Hydroxide | | 2.8 | | 2.2 | 2.9 | 0.6 | | | | 0.2 |
| Natrosol 250 HHR | Hydroxymethyl cellulose | 0.3 | | | | | | | | | |
| (Aqualon) | | | | | | | | | | | |
| Neo Heliopan ® AP | Dinatrium-Phenyl- | | 22.0 | | | | | | | | |
| (Symrise), 15% as | dibenzimidazoltetra- | | | | | | | | | | |
| sodium salt | sulfonat | | | | | | | | | | |
| Neo Heliopan ® AP | Dinatrium-Phenyl- | | | 22.0 | | | | | | | |
| (Symrise), 10% | dibenzimidazoltetra- | | | | | | | | | | |
| aqueous solution | sulfonat | | | | | | | | | | |
| neutralised with | | | | | | | | | | | |
| NaOH) | | | | | | | | | | | |
| Neo Heliopan ® AV | Ethylhexylmethoxy- | | | | 5.0 | 6.0 | 2.0 | | | | |
| (Symrise) | cinnamat | | | | | | | | | | |
| Neo Heliopan ® BB | Benzophenone-3 | 1.0 | | | | | | | | | |
| (Symrise) | | | | | | | | | | | |
| Neo Heliopan ® 303 | Octocrylene | 7.0 | | | | | | | | | |
| (Symrise) | | | | | | | | | | | |
| Neo Heliopan ® 357 | Butyl Methoxydibenzoyl- | | 2.0 | | 1.5 | 1.5 | 1.5 | 0.5 | 0.5 | | |
| (Symrise) | methane | | | | | | | | | | |
| Neo Heliopan ® | Isoamyl-p-methoxy- | | | | 5.0 | | 6.0 | | 2.0 | | |
| E 1000 (Symrise) | cinnamate | | | | | | | | | | |
| Neo Heliopan ® | Homosalate | | 5.0 | | | | | | | | |
| HMS (Symrise) | | | | | | | | | | | |
| Neo Heliopan ® | Phenylbenzimidazol | | | 33.3 | 10.0 | 13.3 | | 3.3 | | | |
| Hydro | sulfonic acid | | | | | | | | | | |
| (15% aqueous | | | | | | | | | | | |
| solution neutralised | | | | | | | | | | | |
| with NaOH) | | | | | | | | | | | |
| (Symrise) | | | | | | | | | | | |
| Neo Heliopan ® MA | Menthylanthranilate | 3.0 | | | | | | | | | |
| (Symrise) | | | | | | | | | | | |
| Neo Heliopan ® | 4-Methylbenzyliden- | | | | 2.0 | 4.0 | 3.0 | | | | |
| MBC (Symrise) | campher | | | | | | | | | | |
| Neo Heliopan ® OS | Ethylhexylsalicylate | 1.0 | | | | | | | | | |
| (Symrise) | | | | | | | | | | | |
| Neo PCL wssl. N | Trideceth-9, PEG-5 | | | 1.0 | 1.5 | | | 1.5 | | | |
| (Symrise) | Ethylhexanoate | | | | | | | | | | |
| Parfümöl | Parfum (Fragrance) | 0.3 | 0.3 | 0.3 | 0.3 | 0.4 | 0.2 | 0.5 | 0.4 | 0.3 | 0.3 |
| (Symrise) | | | | | | | | | | | |
| Pemulen TR 2 | Acrylates/C10-30 Alkyl | | | | | | 0.2 | | | | |
| (Novion) | Acrylate Crosspolymer | | | | | | | | | | |
| Polymer JR 400 | Polyquaternium-10 | | | | | | | 0.4 | | | |
| 1,2-Propylenglycol | Propylene Glycol | | | | | | | | | | 5.0 |
| Simagel M | Quaternium-18-hectorite | 1.0 | | | | | | | | | |
| Solubilizer (Symrise) | PEG 40 Hydrogenated | | | | | | | 3.0 | | | |
| | Castor Oil, Trideceth-9, | | | | | | | | | | |
| | Propyleneglycol, Water | | | | | | | | | | |
| Symdiol 68 | 1,2 Hexanediol, | | 0.5 | | | | | | | | 1.0 |
| | Caprylylglycol | | | | | | | | | | |
| Texapon N 70 | Sodium Laureth Sulfate | | | | | | 0.5 | | | | |
| (Cognis) | | | | | | | | | | | |
| Texapon NSO BZ | Sodium Laureth Sulfate | | | | | | | 27.0 | | | |
| (Cognis) | | | | | | | | | | | |
| Zinc Oxide neutral | Zinc Oxide | 5.0 | | | | | | | | | |
| (Symrise) | | | | | | | | | | | |
| Veegum ultra ® | Magnesium Aluminium | 1.0 | | | | | | | | | |
| (Vanderbilt) | sulfate | | | | | | | | | | |
| Vitamin A Palmitat | Retinyl Palmitat | | | | | 0.1 | | | | 0.1 | |
| Witch Hazel | Hamamelis Virginiana | | | | | | | 1.0 | | | |
| Distillate (Symrise) | (Witch Hazel) | | | | | | | | | | |
| Water, distilled | Aqua (Water) | ad | ad | ad | ad | ad | ad | ad | ad | ad | ad |
| | | 100 | 100 | 100 | 100 | 100 | 100 | 100 | 100 | 100 | 100 |

Preparation 1: “Oil-in-water” emulsion with UV-A/B-broadband protection.
Preparation 2: “Oil-in-water” emulsion with UV-A/B broadband protection
Preparation 3: Sun spray with UV-A/B broadband protection with low oil content
Preparation 4: Skin-lightening balm with UV-A/UV-B protection
Preparation 5: Skin-lightening aerosol foam with UV-B/UV-A protection
Preparation 6: Skin-lightening non-aerosol foam
Preparation 7: Shampoo with skin-lightening properties
Preparation 8: Skin-lightening hair conditioner with UV-B/UV-A protection
Preparation 9: Skin-lightening moisturizing cream O/W
Preparation 10: Skin-lightening face cream O/W

TABLE 4
Cosmetic and pharmaceutical, in particular dermatological preparations
comprising styrylresorcinol of formula 3 (CARN: 85-27-8; 4-(1-phenylethyl)-1,3-
dihydroxybenzene) and one or more skin and/or hair lightening and/or senile
reducing compounds of constituent b) selected from the group (ii) phenolic
derivatives and plant extracts with an amount of phenolic derivatives.

| RAW MATERIAL NAME | | | | | | | | | | | |
|---|---|---|---|---|---|---|---|---|---|---|---|
| (MANUFACTURE) | INCI | 1 | 2 | 3 | 4 | 5 | 6 | 7 | 8 | 9 | 10 |
| Skin lightener | | | | | | | | | | | |
| 4-(1-Phenylethyl)- | Phenylethyl Resorcinol | 0.10 | 0.25 | 0.50 | 0.75 | 1.00 | 2.00 | 0.10 | 0.50 | 1.00 | 0.5 |
| 1,3- | | | | | | | | | | | |
| dihydroxybenzene | | | | | | | | | | | |
| (CARN: 85-278) | | | | | | | | | | | |
| 4-Hydroxyphenyl-β- | Arbutine | | | | | 1.00 | | | | | |
| D-Glucopyranoside | | | | | | | | | | | |
| Hydrochinon | Hydroquinone | | | 1.00 | | | | | 0.50 | | 1.50 |
| 1,3-Benzoldiol | Resorcinol | | 2.00 | | | | | 1.00 | | | |
| 1,3-Dihydroxy-4-n- | 4-Butylresorcinol | | | | 1.50 | | | | | 0.50 | |
| Butylbenzol | | | | | | | | | | | |
| Bärentraubenextrakt | Bearberry Extract | | | 1.50 | | | | | | 0.50 | |
| Kiefernextrakt | Pinus Extract | | 5.00 | | | | | 0.50 | | | 1.00 |
| Maulbeerextrakt | Mulberry Extract | 2.00 | | | | | | | | | |
| Sojabohnenextrakt | Soybean Extract | | | | | | 1.00 | | 2.00 | 1.00 | |
| Brotfruchtextrakt | Artocarpus extract | | | | | 1.00 | | | | | |
| Sü [symbol] holzwurzel | Licorice extract | | | 1.50 | | | | | 0.50 | | 1.00 |
| Extract | | | | | | | | | | | |
| Oil components | | | | | | | | | | | |
| Aloe Vera Gel | Water (Aqua). | 0.05 | 0.1 | | | | | | | | 0.1 |
| Konzentrat 10/1 | Aloe Barbadensis | | | | | | | | | | |
| (Symrise) | Leaf Extract | | | | | | | | | | |
| Abil 100 ® | Dimethicone | 1.0 | 0.3 | | | | 1.0 | | | 2.0 | 0.3 |
| (Goldschmidt) | | | | | | | | | | | |
| Ascorbyl-6 Palmitat | Ascorbyl Palmitate | 0.1 | | 0.2 | | | | | 0.1 | | |
| Cetiol OE (Cognis) | Dicaprylyl Ether | | | | | | 3.0 | | | | |
| Corapan TQ ® | Diethylhexyl-2,6- | | 2.0 | | | | | | | | |
| (Symrise) | Naphtalate | | | | | | | | | | |
| Dragoxat EH | Ethylhexyl | | | 1.0 | 1.0 | | | | | | |
| (Symrise) | Ethylhexanoate | | | | | | | | | | |
| Isoadipate (Symrise) | Diisopropyl Adipate | | | | 1.0 | | | | | | |
| Isopropylmyristat | Isopropyl Myristate | | | | | | | | | | 4.0 |
| (Symrise) | | | | | | | | | | | |
| Isodragol (Symrise) | Triisononanoin | | | | | | | | | 6.0 | |
| Neutralöl (Symrise) | Caprylic/Capric/ | 4.0 | 2.0 | | | 2.0 | | | | | |
| | Triglyceride | | | | | | | | | | |
| Paraffinöl | Mineral Oil | | | | | | | | | | 4.0 |
| PCL Liquid 100 | Cetearyl Octanoate | 4.0 | 3.0 | | | | | | 3.0 | | |
| (Symrise) | | | | | | | | | | | |
| Tegosoft TN ® | C12-C15 Alkylbenzoate | | | | 4.0 | 2.0 | | | | | |
| (Goldschmidt) | | | | | | | | | | | |
| Further ingredients | | | | | | | | | | | |
| Aloe Vera Gel | Water (Aqua). | 0.05 | 0.1 | | | | | | | | 0.1 |
| Konzentrat 10/1 | Aloe Barbadensis | | | | | | | | | | |
| (Symrise) | Leaf Extract | | | | | | | | | | |
| Arlypon F | Laureth-2 | | | | | | | 2.0 | | | |
| alpha-Bisabolol | Bisabolol | | 0.1 | | 0.2 | 0.1 | 0.1 | 0.1 | | | |
| (Symrise) | | | | | | | | | | | |
| Ascorbyl-6 Palmitat | Ascorbyl Palmitate | 0.1 | | 0.2 | | | | | 0.1 | | |
| 1,3-Butylenglycol | 1,3-Butylenglycol | | 3.0 | | | | | | | | |
| Carbopol 2050 ® | Carbomer | | 0.2 | | 0.5 | 0.1 | | | | | |
| (B. F. Goodrich) | | | | | | | | | | | |
| Ceramid Bio 391 | Ceramid | 0.3 | | | | | | | | | 0.1 |
| (Symrise) | | | | | | | | | | | |
| Citric Acid | Citric Acid | | | | | | | | 0.1 | 0.3 | |
| Copherol 1250 ® | Tocopherol acetate | | 0.5 | | 0.5 | 0.5 | 0.5 | | | | |
| (Cognis) | | | | | | | | | | | |
| Crinipan ® AD | Climbazol | | | | | | | 0.5 | | | |
| (Symrise) | | | | | | | | | | | |
| Dehyquart SP | Quaternium-52 | | | | | | | | 0.5 | | |
| Dehyton K | Cocamidopropyl Betaine | | | | | | | 12.0 | | | |
| Diethylenetriamine | Pentetic Acid | 0.05 | 0.1 | | | | | | | | 0.05 |
| Pentaacetic acid | | | | | | | | | | | |
| Dow Corning ® 193 | Dimethicon-Polyol | | | 1.0 | | | | | | | |
| (Dow corning) | | | | | | | | | | | |
| D-Panthenol (BASF) | Panthenol | | | 0.5 | | | 0.5 | 0.4 | | | |
| Dragocid Liquid | Phenoxyethanol (and) | 0.8 | | 0.8 | 0.8 | 0.8 | 0.8 | 0.5 | 0.8 | 0.8 | |
| (Symrise) | Methylparaben (and) | | | | | | | | | | |
| | Ethylparaben (and) | | | | | | | | | | |
| | Butylparaben (and) | | | | | | | | | | |
| | Propylparaben (and) | | | | | | | | | | |
| | Isobutylparaben | | | | | | | | | | |
| Dragophos S | Sodium Dihydroxycetyl | | | | | | | | | 2.0 | |
| (Symrise) | Phosphate | | | | | | | | | | |
| Dracorin CE | Glyceryl Stearate/Citrate | | | | | | | | | 1.0 | |
| Dracorin GMS | Glyceryl Stearate | 2.0 | 2.0 | | | 2.0 | | | | 2.0 | 3.0 |
| (Symrise) | | | | | | | | | | | |
| Dracorin 100 s.e. P | Glyceryl Stearate, PEG- | 3.0 | | | | | | | | | 8.0 |
| (Symrise) | 100 Stearate | | | | | | | | | | |
| Edeta BD ® (BASF) | Dinatrium-EDTA | 0.1 | 0.1 | | 0.1 | 0.1 | 0.1 | | | | |
| Emulgin B2 ® | Ceteareth-20 | 1.0 | | | | | | | 0.7 | | |
| (Cognis) | | | | | | | | | | | |
| Emulsiphos | Potassium | | 1.5 | | | 1.5 | | | | | |
| (Symrise) | Cetylphosphate, | | | | | | | | | | |
| | Hydrogenated Palm | | | | | | | | | | |
| | Glycerides | | | | | | | | | | |
| Ethyl alcohol (96%) | Ethyl alcohol | | | 13.0 | 5.0 | | | | | | |
| Extrapon Kamille | Water (Aqua), Propylene | | | 1.0 | | | | | | | |
| (Symrise) | Glycol, Butylene Glycol, | | | | | | | | | | |
| | Chamomilla Recltita | | | | | | | | | | |
| | (Matricaria) Flower | | | | | | | | | | |
| | Extract, Glucose, | | | | | | | | | | |
| | Bisabolol | | | | | | | | | | |
| Extrapon | Glycerin, Water (Aqua), | | | 1.0 | | | | | | | |
| Hamamelis | Hamamelis Virginiana | | | | | | | | | | |
| (Symrise) | (Witch Hazel) | | | | | | | | | | |
| | Bark/Leaves | | | | | | | | | | |
| Frescolat ML | Menthyl Lactate | | | | 0.5 | | | | | | |
| Glycerin 99% | Glycerin | 3.0 | | 4.5 | | 3.0 | 4.0 | | | | |
| Hydrolite-5 | Pentylene Glycol | | | | 4.5 | | 5.0 | | | 3.0 | |
| (Symrise) | | | | | | | | | | | |
| Keltrol T ® (Kelco) | Xanthan Gum | | 0.2 | 0.2 | 0.3 | | | | | | |
| Lanette E ® (Cognis) | Natriumcetearylsulfat | | 0.7 | | | | | | | | |
| Lanette O ® (Cognis) | Cetaeryl Alcohol | 1.1 | | | | | | | 2.5 | | |
| Lanette 16 ® | Cetylalkohol | | 1.2 | | | 0.5 | | | | | 2.0 |
| (Cognis) | | | | | | | | | | | |
| Lanette 18 (Care | Stearyl Alcohol | | | | | | | | | 4.5 | |
| Chemicals) | | | | | | | | | | | |
| Lara Care A-200 | Galactoarabinan | | | | | | 0.2 | | | | |
| (Rahn) | | | | | | | | | | | |
| Magnesium L- | Magnesium Ascorbyl | 0.1 | | 0.05 | | 0.3 | | | | | 0.1 |
| Ascorbyl-2- | Palmitate | | | | | | | | | | |
| phosphate | | | | | | | | | | | |
| NaOH 10% | Sodium hydroxide | | 2.8 | | 2.2 | 2.9 | 0.6 | | | | 0.2 |
| aqueous solution | | | | | | | | | | | |
| Natrosol 250 HHR | Hydroxymethyl cellulose | 0.3 | | | | | | | | | |
| (Aqualon) | | | | | | | | | | | |
| Neo Heliopan ® AP | Dinatrium-Phenyl- | | 22.0 | | | | | | | | |
| (Symrise), 15% as | dibenzimidazoltetra- | | | | | | | | | | |
| sodium salt | sulfonat | | | | | | | | | | |
| Neo Heliopan ® AP | Dinatrium-Phenyl- | | | 22.0 | | | | | | | |
| (Symrise), 10% | dibenzimidazoltetra- | | | | | | | | | | |
| aqueous solution | sulfonat | | | | | | | | | | |
| neutralised with | | | | | | | | | | | |
| NaOH) | | | | | | | | | | | |
| Neo Heliopan ® AV | Ethylhexylmethoxy- | | | | 5.0 | 6.0 | 2.0 | | | | |
| (Symrise) | cinnamat | | | | | | | | | | |
| Neo Heliopan ® BB | Benzophenone-3 | 1.0 | | | | | | | | | |
| (Symrise) | | | | | | | | | | | |
| Neo Heliopan ® 303 | Octocrylene | 7.0 | | | | | | | | | |
| (Symrise) | | | | | | | | | | | |
| Neo Heliopan ® 357 | Butyl Methoxydibenzoyl- | | 2.0 | | 1.5 | 1.5 | 1.5 | 0.5 | 0.5 | | |
| (Symrise) | methane | | | | | | | | | | |
| Neo Heliopan ® | Isoamyl-p-methoxy- | | | | 5.0 | | 6.0 | | 2.0 | | |
| E 1000 (Symrise) | cinnamate | | | | | | | | | | |
| Neo Heliopan ® | Homosalate | | 5.0 | | | | | | | | |
| HMS (Symrise) | | | | | | | | | | | |
| Neo Heliopan ® | Phenylbenzimidazol | | | 33.3 | 10.0 | 13.3 | | 3.3 | | | |
| Hydro | sulfonic acid | | | | | | | | | | |
| (15% aqueous | | | | | | | | | | | |
| solution neutralised | | | | | | | | | | | |
| with NaOH) | | | | | | | | | | | |
| (Symrise) | | | | | | | | | | | |
| Neo Heliopan ® MA | Menthylanthranilate | 3.0 | | | | | | | | | |
| (Symrise) | | | | | | | | | | | |
| Neo Heliopan ® | 4-Methylbenzyliden- | | | | 2.0 | 4.0 | 3.0 | | | | |
| MBC (Symrise) | campher | | | | | | | | | | |
| Neo Heliopan ® OS | Ethylhexylsalicylate | 1.0 | | | | | | | | | |
| (Symrise) | | | | | | | | | | | |
| Neo PCL water | Trideceth-9, PEG-5 | | | 1.0 | 1.5 | | | 1.5 | | | |
| soluble N | Ethylhexanoate | | | | | | | | | | |
| (Symrise) | | | | | | | | | | | |
| Perfume oil | Parfum (Fragrance) | 0.3 | 0.3 | 0.3 | 0.3 | 0.4 | 0.2 | 0.5 | 0.4 | 0.3 | 0.3 |
| (Symrise) | | | | | | | | | | | |
| Pemulen TR 2 | Acrylates/C10-30 Alkyl | | | | | | 0.2 | | | | |
| (Novion) | Acrylate Crosspolymer | | | | | | | | | | |
| Polymer JR 400 | Polyquaternium-10 | | | | | | | 0.4 | | | |
| 1,2-Propylenglycol | Propylene Glycol | | | | | | | | | | 5.0 |
| Simagel M | Quaternium-18-hectorite | 1.0 | | | | | | | | | |
| Solubilizer (Symrise) | PEG 40 Hydrogenated | | | | | | | 3.0 | | | |
| | Castor Oil, Trideceth-9, | | | | | | | | | | |
| | Propyleneglycol, Water | | | | | | | | | | |
| Symdiol 68 | 1,2 Hexanediol, | | 0.5 | | | | | | | | 1.0 |
| | Caprylylglycol | | | | | | | | | | |
| Texapon N 70 | Sodium Laureth Sulfate | | | | | | 0.5 | | | | |
| (Cognis) | | | | | | | | | | | |
| Texapon NSO BZ | Sodium Laureth Sulfate | | | | | | | 27.0 | | | |
| (Cognis) | | | | | | | | | | | |
| Zinc Oxide neutral | Zinc Oxide | 5.0 | | | | | | | | | |
| (Symrise) | | | | | | | | | | | |
| Veegum ultra ® | Magnesium Aluminium | 1.0 | | | | | | | | | |
| (Vanderbilt) | sulfate | | | | | | | | | | |
| Vitamin A Palmitat | Retinyl Palmitat | | | | | 0.1 | | | | 0.1 | |
| Witch Hazel | Hamamelis Virginiana | | | | | | | 1.0 | | | |
| Distillate (Symrise) | (Witch Hazel) | | | | | | | | | | |
| Water, distilled | Aqua (Water) | ad | ad | ad | ad | ad | ad | ad | ad | ad | ad |
| | | 100 | 100 | 100 | 100 | 100 | 100 | 100 | 100 | 100 | 100 |

Preparation 1: “Oil-in-water” emulsion with UV-A/B-broadband protection.
Preparation 2: “Oil-in-water” emulsion with UV-A/B broadband protection
Preparation 3: Sun spray with UV-A/B broadband protection with low oil content
Preparation 4: Skin-lightening balm with UV-A/UV-B protection
Preparation 5: Skin-lightening aerosol foam with UV-B/UV-A protection
Preparation 6: Skin-lightening non-aerosol foam
Preparation 7: Shampoo with skin-lightening properties
Preparation 8: Skin-lightening hair conditioner with UV-B/UV-A protection
Preparation 9: Skin-lightening moisturizing cream O/W
Preparation 10: Skin-lightening face cream O/W

TABLE 5
Cosmetic and pharmaceutical, in particular dermatological preparations
comprising styrylresorcinol of formula 3 (CARN: 85-27-8; 4-(1-phenylethyl)-1,3-
dihydroxybenzene) and one or more skin and/or hair lightening and/or senile reducing
compounds of constituent b) selected from the group (iii) organic acid derivatives.

| RAW MATERIAL | | | | | | | | | | | |
|---|---|---|---|---|---|---|---|---|---|---|---|
| NAME | | | | | | | | | | | |
| (MANUFACTURER) | INCI | 1 | 2 | 3 | 4 | 5 | 6 | 7 | 8 | 9 | 10 |
| Skin lightener | | | | | | | | | | | |
| 4-(1-Phenylethyl)- | Phenylethyl Resorcinol | 0.10 | 0.25 | 0.50 | 0.75 | 1.00 | 2.00 | 0.10 | 0.50 | 1.00 | 0.5 |
| 1,3- | | | | | | | | | | | |
| dihydroxybenzene | | | | | | | | | | | |
| (CARN: 85-278) | | | | | | | | | | | |
| Azelaic acid | Azelaic acid | 2.00 | | | | | | | | | 1.00 |
| 9-Octadecenedioic | 9-Octadecenedioic | | | | | 1.00 | | | | 0.50 | |
| acid | acid | | | | | | | | | | |
| alpha-Lipoic acid | alpha-Lipoic acid | | 0.75 | | | | | | 0.50 | | |
| Retinoic acid | Retinoic acid | | | | | | 0.10 | | | | |
| Niacinamide | Niacinamide | | | 1.50 | | | | 1.00 | | | |
| Undecylenoyl | Undecylenoyl | | | | 1.50 | | | | | | |
| Phenylalanine | Phenylalanine | | | | | | | | | | |
| Oil components | | | | | | | | | | | |
| Aloe Vera Gel | Water (Aqua). | 0.05 | 0.1 | | | | | | | | 0.1 |
| conzentrat 10/1 | Aloe Barbadensis | | | | | | | | | | |
| (Symrise) | Leaf Extract | | | | | | | | | | |
| Abil 100 ® | Dimethicone | 1.0 | 0.3 | | | | 1.0 | | | 2.0 | 0.3 |
| (Goldschmidt) | | | | | | | | | | | |
| Cetiol OE (Cognis) | Dicaprylyl Ether | | | | | | 3.0 | | | | |
| Corapan TQ ® | Diethylhexyl-2,6- | | 2.0 | | | | | | | | |
| (Symrise) | Naphtalate | | | | | | | | | | |
| Dragoxat EH | Ethylhexyl | | | 1.0 | 1.0 | | | | | | |
| (Symrise) | Ethylhexanoate | | | | | | | | | | |
| Isoadipate (Symrise) | Diisopropyl Adipate | | | | 1.0 | | | | | | |
| Isopropylmyristat | Isopropyl Myristate | | | | | | | | | | 4.0 |
| (Symrise) | | | | | | | | | | | |
| Isodragol (Symrise) | Triisononanoin | | | | | | | | | 6.0 | |
| Neutralöl (Symrise) | Caprylic/Capric/ | 4.0 | 2.0 | | | 2.0 | | | | | |
| | Triglyceride | | | | | | | | | | |
| Paraffin oil | Mineral Oil | | | | | | | | | | 4.0 |
| PCL Liquid 100 | Cetearyl Octanoate | 4.0 | 3.0 | | | | | | 3.0 | | |
| (Symrise) | | | | | | | | | | | |
| Tegosoft TN ® | C12-C15 Alkylbenzoate | | | | 4.0 | 2.0 | | | | | |
| (Goldschmidt) | | | | | | | | | | | |
| Further ingredients | | | | | | | | | | | |
| Arlypon F | Laureth-2 | | | | | | | 2.0 | | | |
| alpha-Bisabolol | Bisabolol | | 0.1 | | 0.2 | 0.1 | 0.1 | 0.1 | | | |
| (Symrise) | | | | | | | | | | | |
| Ascorbyl-6 Palmitat | Ascorbyl Palmitate | 0.1 | | 0.2 | | | | | 0.1 | | |
| 1,3-Butylenglycol | 1,3-Butylenglycol | | 3.0 | | | | | | | | |
| Carbopol 2050 ® | Carbomer | | 0.2 | | 0.5 | 0.1 | | | | | |
| (B. F. Goodrich) | | | | | | | | | | | |
| Ceramid Bio 391 | Ceramid | 0.3 | | | | | | | | | 0.1 |
| (Symrise) | | | | | | | | | | | |
| Citric Acid | Citric Acid | | | | | | | | 0.1 | 0.3 | |
| Copherol 1250 ® | Tocopherol acetate | | 0.5 | | 0.5 | 0.5 | 0.5 | | | | |
| (Cognis) | | | | | | | | | | | |
| Crinipan ® AD | Climbazol | | | | | | | 0.5 | | | |
| (Symrise) | | | | | | | | | | | |
| Dehyquart SP | Quaternium-52 | | | | | | | | 0.5 | | |
| Dehyton K | Cocamidopropyl Bataine | | | | | | | 12.0 | | | |
| Diethylenetriamine | Pentetic Acid | 0.05 | 0.1 | | | | | | | | 0.05 |
| Pentaacetsäure | | | | | | | | | | | |
| Dow Corning ® 193 | Dimethicon-Polyol | | | 1.0 | | | | | | | |
| (Dow corning) | | | | | | | | | | | |
| D-Panthenol (BASF) | Panthenol | | | 0.5 | | | 0.5 | 0.4 | | | |
| Dragocid Liquid | Phenoxyethanol (and) | 0.8 | | 0.8 | 0.8 | 0.8 | 0.8 | 0.5 | 0.8 | 0.8 | |
| (Symrise) | Methylparaben (and) | | | | | | | | | | |
| | Ethylparaben (and) | | | | | | | | | | |
| | Butylparaben (and) | | | | | | | | | | |
| | Propylparaben (and) | | | | | | | | | | |
| | Isobutylparaben | | | | | | | | | | |
| Dragophos S | Sodium Dihydroxycetyl | | | | | | | | | 2.0 | |
| (Symrise) | Phosphate | | | | | | | | | | |
| Dracorin CE | Glyceryl Stearate/Citrate | | | | | | | | | 1.0 | |
| Dracorin GMS | Glyceryl Stearate | 2.0 | 2.0 | | | 2.0 | | | | 2.0 | 3.0 |
| (Symrise) | | | | | | | | | | | |
| Dracorin 100 s.e. P | Glyceryl Stearate, PEG- | 3.0 | | | | | | | | | 8.0 |
| (Symrise) | 100 Stearate | | | | | | | | | | |
| Edeta BD ® (BASF) | Dinatrium-EDTA | 0.1 | 0.1 | | 0.1 | 0.1 | 0.1 | | | | |
| Emulgin B2 ® | Ceteareth-20 | 1.0 | | | | | | | 0.7 | | |
| (Cognis) | | | | | | | | | | | |
| Emulsiphos | Potassium | | 1.5 | | | 1.5 | | | | | |
| (Symrise) | Cetylphosphate, | | | | | | | | | | |
| | Hydrogenated Palm | | | | | | | | | | |
| | Glycerides | | | | | | | | | | |
| Ethyl alcoholl (96%) | Ethyl alcohol | | | 13.0 | 5.0 | | | | | | |
| Extrapon Kamille | Water (Aqua), Propylene | | | 1.0 | | | | | | | |
| (Symrise) | Glycol, Butylene Glycol, | | | | | | | | | | |
| | Chamomilla Recitita | | | | | | | | | | |
| | (Matricaria) Flower | | | | | | | | | | |
| | Extract, Glucose, | | | | | | | | | | |
| | Bisabolol | | | | | | | | | | |
| Extrapon | Glycerin, Water (Aqua), | | | 1.0 | | | | | | | |
| Hamamelis | Hamamelis Virginiana | | | | | | | | | | |
| (Symrise) | (Witch Hazel) | | | | | | | | | | |
| | Bark/Leaves | | | | | | | | | | |
| Frescolat ML | Menthyl Lactate | | | | 0.5 | | | | | | |
| Glycerin 99% | Glycerin | 3.0 | | 4.5 | | 3.0 | 4.0 | | | | |
| Hydrolite-5 | Pentylene Glycol | | | | 4.5 | | 5.0 | | | 3.0 | |
| (Symrise) | | | | | | | | | | | |
| Keltrol T ® (Kelco) | Xanthan Gum | | 0.2 | 0.2 | 0.3 | | | | | | |
| Lanette E ® (Cognis) | Natriumcetearylsulfat | | 0.7 | | | | | | | | |
| Lanette O ® (Cognis) | Cetaeryl Alcohol | 1.1 | | | | | | | 2.5 | | |
| Lanette 16 ® | Cetylalkohol | | 1.2 | | | 0.5 | | | | | 2.0 |
| (Cognis) | | | | | | | | | | | |
| Lanette 18 (Care | Stearyl Alcohol | | | | | | | | | 4.5 | |
| Chemicals) | | | | | | | | | | | |
| Lara Care A-200 | Galactoarabinan | | | | | | 0.2 | | | | |
| (Rahn) | | | | | | | | | | | |
| Magnesium L- | Magnesium Ascorbyl | 0.1 | | 0.05 | | 0.3 | | | | | 0.1 |
| Ascorbyl-2- | Palmitate | | | | | | | | | | |
| phosphate | | | | | | | | | | | |
| NaOH 10% | Sodium hydroxide | | 2.8 | | 2.2 | 2.9 | 0.6 | | | | 0.2 |
| aqueous solution | | | | | | | | | | | |
| Natrosol 250 HHR | Hydroxymethyl cellulose | 0.3 | | | | | | | | | |
| (Aqualon) | | | | | | | | | | | |
| Neo Heliopan ® AP | Dinatrium-Phenyl- | | 22.0 | | | | | | | | |
| (Symrise), 15% as | dibenzimidazoltetra- | | | | | | | | | | |
| sodium salt | sulfonat | | | | | | | | | | |
| Neo Heliopan ® AP | Dinatrium-Phenyl- | | | 22.0 | | | | | | | |
| (Symrise), 10% | dibenzimidazoltetra- | | | | | | | | | | |
| aqueous solution | sulfonat | | | | | | | | | | |
| neutralised with | | | | | | | | | | | |
| NaOH) | | | | | | | | | | | |
| Neo Heliopan ® AV | Ethylhexylmethoxy- | | | | 5.0 | 6.0 | 2.0 | | | | |
| (Symrise) | cinnamat | | | | | | | | | | |
| Neo Heliopan ® BB | Benzophenone-3 | 1.0 | | | | | | | | | |
| (Symrise) | | | | | | | | | | | |
| Neo Heliopan ® 303 | Octocrylene | 7.0 | | | | | | | | | |
| (Symrise) | | | | | | | | | | | |
| Neo Heliopan ® 357 | Butyl Methoxydibenzoyl- | | 2.0 | | 1.5 | 1.5 | 1.5 | 0.5 | 0.5 | | |
| (Symrise) | methane | | | | | | | | | | |
| Neo Heliopan ® | Isoamyl-p-methoxy- | | | | 5.0 | | 6.0 | | 2.0 | | |
| E 1000 (Symrise) | cinnamate | | | | | | | | | | |
| Neo Heliopan ® | Homosalate | | 5.0 | | | | | | | | |
| HMS (Symrise) | | | | | | | | | | | |
| Neo Heliopan ® | Phenylbenzimidazol | | | 33.3 | 10.0 | 13.3 | | 3.3 | | | |
| Hydro | sulfonic acid | | | | | | | | | | |
| (15% aqueous | | | | | | | | | | | |
| solution neutralised | | | | | | | | | | | |
| with NaOH) | | | | | | | | | | | |
| (Symrise) | | | | | | | | | | | |
| Neo Heliopan ® MA | Menthylanthranilate | 3.0 | | | | | | | | | |
| (Symrise) | | | | | | | | | | | |
| Neo Heliopan ® | 4-Methylbenzyliden- | | | | 2.0 | 4.0 | 3.0 | | | | |
| MBC (Symrise) | campher | | | | | | | | | | |
| Neo Heliopan ® OS | Ethylhexylsalicylate | 1.0 | | | | | | | | | |
| (Symrise) | | | | | | | | | | | |
| Neo PCL water | Trideceth-9, PEG-5 | | | 1.0 | 1.5 | | | 1.5 | | | |
| soluble N | Ethylhexanoate | | | | | | | | | | |
| (Symrise) | | | | | | | | | | | |
| Perfume oil | Parfum (Fragrance) | 0.3 | 0.3 | 0.3 | 0.3 | 0.4 | 0.2 | 0.5 | 0.4 | 0.3 | 0.3 |
| (Symrise) | | | | | | | | | | | |
| Pemulen TR 2 | Acrylates/C10-30 Alkyl | | | | | | 0.2 | | | | |
| (Novion) | Acrylate Crosspolymer | | | | | | | | | | |
| Polymer JR 400 | Polyquaternium-10 | | | | | | | 0.4 | | | |
| 1,2-Propylenglycol | propilene Glycol | | | | | | | | | | 5.0 |
| Simagel M | Quaternium-18-hectorite | 1.0 | | | | | | | | | |
| Solubilizer (Symrise) | PEG 40 Hydrogenated | | | | | | | 3.0 | | | |
| | Castor Oil, Trideceth-9, | | | | | | | | | | |
| | Propyleneglycol, Water | | | | | | | | | | |
| Symdiol 68 | 1,2 Hexanediol, | | 0.5 | | | | | | | | 1.0 |
| | Caprylylglycol | | | | | | | | | | |
| Texapon N 70 | Sodium Laureth Sulfate | | | | | | 0.5 | | | | |
| (Cognis) | | | | | | | | | | | |
| Texapon NSO BZ | Sodium Laureth Sulfate | | | | | | | 27.0 | | | |
| (Cognis) | | | | | | | | | | | |
| Zinc Oxide neutral | Zinc Oxide | 5.0 | | | | | | | | | |
| (Symrise) | | | | | | | | | | | |
| Veegum ultra ® | Magnesium Aluminium | 1.0 | | | | | | | | | |
| (Vanderbilt) | sulfate | | | | | | | | | | |
| Vitamin A Palmitat | Retinyl Palmitat | | | | | 0.1 | | | | 0.1 | |
| Witch Hazel | Hamamelis Virginiana | | | | | | | 1.0 | | | |
| Distillate (Symrise) | (Witch Hazel) | | | | | | | | | | |
| Wasser, distilled | Aqua (Water) | ad 100 | ad 100 | ad 100 | ad 100 | ad 100 | ad 100 | ad 100 | ad 100 | ad 100 | ad 100 |

Preparation 1: “Oil-in-water” emulsion with UV-A/B-broadband protection.
Preparation 2: “Oil-in-water” emulsion with UV-A/B broadband protection
Preparation 3: Sun spray with UV-A/B broadband protection with low oil content
Preparation 4: Skin-lightening balm with UV-A/UV-B protection
Preparation 5: Skin-lightening aerosol foam with UV-B/UV-A protection
Preparation 6: Skin-lightening non-aerosol foam
Preparation 7: Shampoo with skin-lightening properties
Preparation 8: Skin-lightening hair conditioner with UV-B/UV-A protection
Preparation 9: Skin-lightening moisturizing cream O/W
Preparation 10: Skin-lightening face cream O/W

### EXAMPLE 3

### Production of Colour-Stable Preparations

Further Examples (According to the Invention and not According to the Invention) of Colour-Stable Preparations Produced in Accordance with the Methods of the Invention of Stabilizing Phenolic Compounds of the General Formula 1 are Described Below:

Table 6 lists by way of example further colour-stable preparations containing diphenols such as, for example, 4-(1-phenylethyl)-1,3-dihydroxybenzene (CARN: 85-27-8; formula 1 or, for example, 4-butylresorcinol (CARN: 18979-61-8). An improvement in the stability, and particularly the colour stability, is achieved through the addition of photoprotective filters, and in particular through the inventive addition of the phenolic compounds of the general formula 1 in a predissolved form in an oil phase additionally containing an photoprotective filter benzophenone-3, it also being possible, in addition, to obtain a further prevention of degradation and of the associated colour changes through the addition of metal chelators and through the adjustment of the pH to values of less than or equal to 5.5 and preferably less than or equal to 4.5.

TABLE 6

| RAW MATERIAL NAME | | | | | | | | | | |
|---|---|---|---|---|---|---|---|---|---|---|
| (MANUFACTURER) | INCI | 1 | 2 | 3 | 4 | 5 | 6 | 7 | 8 | 9 |
| Skin lightener or | | | | | | | | | | |
| mixture containing | | | | | | | | | | |
| skin lightener | | | | | | | | | | |
| 4-(1- | Phenylethylresorcinol | 10.0 | 5.0 | 1.0 | 5.0 | 2.5 | 0.5 | 2.5 | 1.0 | 2.5 |
| Phenylethyl)1,3- | and Ethylhexyl | | | | | | | | | |
| dihydroxybenzene | Isononanoate and | | | | | | | | | |
| and | Benzophenone-3 | | | | | | | | | |
| Dragoxat 89 and | | | | | | | | | | |
| Neo Heliopan BB | | | | | | | | | | |
| Ratio 20:79:1 | | | | | | | | | | |
| 4-Butylresorcinol | 4-Butylresorcinol | | 0.25 | | | | | | 5.0 | |
| and | and Ethylhexyl | | | | | | | | | |
| Dragoxat 89 and | Isononanoate and | | | | | | | | | |
| Neo Heliopan BB | Benzophenone-3 | | | | | | | | | |
| Ratio 20:79:1 | | | | | | | | | | |
| β-Arbutin | Arbutin | | | | 0.5 | | | | 0.2 | |
| Kojic acid | Kojic Acid | | 0.5 | | | | | | | 1.0 |
| Mg ascorbyl- | Magnesium Ascorbyl- | | | | | | | | 3.0 | |
| phosphate | phosphate | | | | | | | | | |
| Liquorice extract | | | | | | | 2.0 | | | |
| Niacinamide | | | | 1.0 | | | | | | |
| Soja extract | | | 1.0 | | | | | | | 1.0 |
| Compounds of the | | | | | | | | | | |
| Formula (II) | | | | | | | | | | |
| Neo Heliopan ® BB | Benzophenone-3 | 0.5 | 0.5 | 0.5 | 0.5 | 0.5 | 0.5 | 0.5 | 0.5 | 0.5 |
| (Symrise) | | | | | | | | | | |
| Oil components | | | | | | | | | | |
| PCL Liquid 100 | Cetearyl Octanoate | 4.0 | 3.0 | | | | | 3.0 | | |
| Corapan TQ ® | Diethylhexyl 1,6- | | 2.0 | | | | | | | |
| (Symrise) | Naphthalate | | | | | | | | | |
| Dragoxat | Ethylhexyl Isononoate | | | 1.0 | 1.0 | | | | | |
| 89 (Symrise) | | | | | | | | | | |
| Isoadipate | Diisopropyl Adipate | | | | 1.0 | | | | | |
| Isopropyl myristate | Isopropyl Myristate | | | | | | | | | 4.0 |
| (Symrise) | | | | | | | | | | |
| Neutral oil (Symrise) | Caprylic/Capric/ | 4.0 | 2.0 | | | 2.0 | | | | |
| | Triglyceride | | | | | | | | | |
| Isodragol (Symrise) | Triisononanoin | | | | | | | | 6.0 | |
| Cetiol OE (Cognis) | Dicaprylyl Ether | | | | | | 3.0 | | | |
| Paraffin oil | Mineral Oil | | | | | | | | | 4.0 |
| Tegosoft TN ® | C12-C15 Alkylbenzoate | | | | 4.0 | 2.0 | | | | |
| (Goldschmidt) | | | | | | | | | | |
| Abil 100 ® | Dimethicone | 1.0 | 0.3 | | | | 1.0 | | 2.0 | 0.3 |
| (Goldschmidt) | | | | | | | | | | |
| Further ingredients | | | | | | | | | | |
| Bentone Gel M IO ® | Mineral Oil and | 3.0 | | | | | | | | |
| (Rheox) | Quaternium-18-hectorite | | | | | | | | | |
| | and Propylene | | | | | | | | | |
| | Carbonate Glyceryl | | | | | | | | | |
| | Stearate and Cetyl | | | | | | | | | |
| | Alcohol | | | | | | | | | |
| alpha-Bisabolol | Bisabolol | | 0.1 | | 0.2 | 0.1 | 0.1 | | | |
| (Symrise) | | | | | | | | | | |
| 1,3-Butylene glycol | 1,3-Butylene Glycol | | 3.0 | | | | | | | |
| Carbopol 2050 ® | Carbomer | | 0.2 | | | 0.10 | | | | |
| (B. F. Goodrich) | | | | | | | | | | |
| Carbopol ETD 2001 | Carbomer | | | | 0.5 | | | | | |
| (Noveon) | | | | | | | | | | |
| Cetiol OE (Cognis) | Dicaprylyl Ether | | | | | | 3.0 | | | |
| Citric Acid | Citric Acid | | | | | | | 0.1 | 0.3 | |
| Copherol 1250 ® | Tocopherol Acetate | | 0.5 | | 0.5 | 0.5 | 0.5 | | | |
| (Cognis) | | | | | | | | | | |
| Dehyquart SP | Quaternium-52 | | | | | | | 0.5 | | |
| Dow Corning ® 193 | Dimethicone Polyol | | | 1.0 | | | | | | |
| (Dow corning) | | | | | | | | | | |
| D-Panthenol (BASF) | Panthenol | | | 0.5 | | | 0.5 | | | |
| Dracorin 100 s.e. | Glyceryl Stearate, PEG- | 3.0 | | | | | | | | |
| | 100 Stearate | | | | | | | | | |
| Dragocid Liquid | Phenoxyethanol (and) | 0.8 | 0.8 | | 0.8 | 0.8 | 0.8 | 0.8 | 0.8 | |
| (Symrise) | Methylparaben (and) | | | | | | | | | |
| | Ethylparaben (and) | | | | | | | | | |
| | Butylparaben (and) | | | | | | | | | |
| | Propylparaben (and) | | | | | | | | | |
| | Isobutylparaben | | | | | | | | | |
| Dragophos S | Sodium Dihydroxycetyl | | | | | | | | 2.0 | |
| (Symrise) | Phosphate | | | | | | | | | |
| Dracorin CE | Glyceryl Stearate/Citrate | | | | | | | | 1.0 | |
| Dracorin GMS | Glyceryl Stearate | 2.0 | 2.0 | | | 2.0 | | | 2.0 | 3.0 |
| (Symrise) | | | | | | | | | | |
| Dracorin 100 s.e. P | Glyceryl Stearate, PEG- | | | | | | | | | 8.0 |
| (Symrise) | 100 Stearate | | | | | | | | | |
| EDTA BD ® (BASF) | Disodium-EDTA | 0.15 | 0.15 | 0.15 | 0.15 | 0.15 | 0.15 | 0.15 | 0.15 | 0.15 |
| Emulgin B2 ® | Ceteareth-20 | 1.0 | | | | | | 0.7 | | |
| (Cognis) | | | | | | | | | | |
| Emulsiphos | Cetyl Phosphate, | | 1.5 | | | 1.5 | | | | |
| (Symrise) | Hydrated Palm | | | | | | | | | |
| | Glycerides | | | | | | | | | |
| Ethanol (96%) | Ethyl Alcohol | | | 13.0 | 5.0 | | | | | |
| Extrapon Aloe Vera | | | | 1.0 | | | | | | |
| (Symrise) | | | | | | | | | | |
| Extrapon Kamille | | | | 1.0 | | | | | | |
| (Symrise) | | | | | | | | | | |
| Extrapon Hamamelis | | | | 1.0 | | | | | | |
| (Symrise) | | | | | | | | | | |
| Glycerin 99% | Glycerin | 3.0 | | 4.5 | | 3.0 | 4.0 | | | |
| Hydrolite-5 | Pentylene Glycol | | | | 4.5 | | 5.0 | | 3.0 | |
| (Symrise) | | | | | | | | | | |
| Keltrol T ® (Calgon) | Xanthan Gum | | 0.2 | 0.2 | 0.3 | | | | | |
| Lanette E ® (Cognis) | Sodium Cetearyl | | 0.7 | | | | | | | |
| | Sulphate | | | | | | | | | |
| Lanette O ® (Cognis) | Cetearyl Alcohol | 1.1 | | | | | | 2.5 | | |
| Lanette 16 ® | Cetyl Alcohol | | 1.2 | | | 0.5 | | | | 2.0 |
| (Cognis) | | | | | | | | | | |
| Lanette 18 (Care | Stearyl Alcohol | | | | | | | | 4.5 | |
| Chemicals) | | | | | | | | | | |
| Lara Care A-200 | Galactoarabinan | | | | | | 0.2 | | | |
| (Rahn) | | | | | | | | | | |
| NaOH 10% | Sodium Hydroxide | | 2.8 | | 2.2 | 2.9 | 0.6 | | | 0.2 |
| aqueous solution | | | | | | | | | | |
| Natrosol 250 HHR | Hydroxymethyl Cellulose | 0.3 | | | | | | | | |
| (Aqualon) | | | | | | | | | | |
| Neo Heliopan ® AP | Disodium Phenyl- | | 22.0 | | | | | | | |
| (Symrise), 15% as | dibenzimidazoletetra- | | | | | | | | | |
| sodium salt | sulphonate | | | | | | | | | |
| Neo Heliopan ® AP | Disodium Phenyl- | | | 22.0 | | | | | | |
| (Symrise), (10% | dibenzimidazoletetra- | | | | | | | | | |
| aqueous solution | sulphonate | | | | | | | | | |
| neutralized with | | | | | | | | | | |
| NaOH) | | | | | | | | | | |
| Neo Heliopan ® AV | Ethylhexyl Methoxy- | | | | 5.0 | 6.0 | 2.0 | | | |
| (Symrise) | cinnamate | | | | | | | | | |
| Neo Heliopan ® 303 | Octocrylene | 7.0 | | | | | | | | |
| (Symrise) | | | | | | | | | | |
| Neo Heliopan ® 357 | Butyl Methoxydibenzoyl- | | 2.0 | | 1.5 | 1.5 | 1.5 | 0.5 | | |
| (Symrise) | methane | | | | | | | | | |
| Neo Heliopan ® | Isoamyl p-Methoxy- | | | | 5.0 | | 6.0 | 2.0 | | |
| E 1000 (Symrise) | cinnamate | | | | | | | | | |
| Neo Heliopan ® | Homosalate | | 5.0 | | | | | | | |
| HMS (Symrise) | | | | | | | | | | |
| Neo Heliopan ® | Phenylbenzimidazole- | | | 33.3 | 10.0 | 13.3 | | | | |
| Hydro | sulphonic Acid | | | | | | | | | |
| (15% aqueous | | | | | | | | | | |
| solution neutralized | | | | | | | | | | |
| with NaOH) | | | | | | | | | | |
| (Symrise) | | | | | | | | | | |
| Neo Heliopan ® MA | Menthyl Anthranilate | 3.0 | | | | | | | | |
| (Symrise) | | | | | | | | | | |
| Neo Heliopan ® | 4-Methylbenzylidene- | | | | 2.0 | 4.0 | 3.0 | | | |
| MBC (Symrise) | camphor | | | | | | | | | |
| Neo Heliopan ® OS | Ethylhexyl Salicylate | 1.0 | | | | | | | | |
| (Symrise) | | | | | | | | | | |
| Neo PCL wssl. N | Trideceth-9, PEG-5 | | | 1.0 | 1.5 | | | | | |
| | Ethylhexanoate | | | | | | | | | |
| Perfume oil | Parfum (Fragrance) | 0.3 | 0.3 | 0.3 | 0.3 | 0.4 | 0.2 | 0.4 | 0.3 | 0.3 |
| Pemulen TR 2 | Acrylates/C10-30 Alkyl | | | | | | 0.2 | | | |
| (Novion) | Acrylate Crosspolymer | | | | | | | | | |
| 1,2-Propylene glycol | Propylene Glycol | | | | | | | | | 5.0 |
| Texapon N 70 | Sodium Laureth | | | | | | 0.5 | | | |
| (Cognis) | Sulphate | | | | | | | | | |
| Zinc Oxide neutral | Zinc Oxide | 5.0 | | | | | | | | |
| (Symrise) | | | | | | | | | | |
| Veegum ultra ® | Magnesium Aluminium | 1.0 | | | | | | | | |
| (Vanderbilt) | Sulphate | | | | | | | | | |
| Water, distilled | Aqua (Water) | ad | ad | ad | ad | ad | ad | ad | ad | ad |
| | | 100 | 100 | 100 | 100 | 100 | 100 | 100 | 100 | 100 |
| pH value | | 4.0 | 5.4 | 4.3 | 5.4 | 5.4 | 5.2 | 5.0 | 4.9 | 4.8 |

Preparation 1: “Oil in water” emulsion with UVA/B broadband protection
Preparation 2: “Oil in water” emulsion with UVA/B broadband protection
Preparation 3: Sun spray with UVA/B broadband protection with low oil content
Preparation 4: Skin-lightening balm with UVA/UVB protection
Preparation 5: Skin-lightening aerosol foam with UVB/UVA protection
Preparation 6: Skin-lightening non-aerosol foam
Preparation 7: Skin-lightening hair conditioner with UVB/UVA protection
Preparation 8: Skin-lightening moisture cream O/W
Preparation 9: Skin-lightening face cream O/W

### EXAMPLE 4

### Examples of Preparations Having a Low Content of Oily Phase (According to the Invention and not According to the Invention)

Cosmetic and/or pharmaceutical preparations which show particularly good results in human in vivo use since they have a content of oily phase which is reduced according to the invention are listed by way of example in the table 7.

TABLE 7

| RAW MATERIAL NAME | | | | | | | | | | | |
|---|---|---|---|---|---|---|---|---|---|---|---|
| (MANUFACTURER) | INCI | 1 | 2 | 3 | 4 | 5 | 6 | 7 | 8 | 9 | 10 |
| Skin lightener | | | | | | | | | | | |
| 4-(1-Phenylethyl)- | | 5.0 | 0.05 | 0.2 | 1.0 | 0.5 | 0.1 | 0.5 | 0.2 | | 0.5 |
| 1,3-benzenediol | | | | | | | | | | | |
| 4-Butylresorcinol | | | 0.05 | | | | | | | 1.0 | |
| beta-Arbutin | Arbutin | | | | 0.5 | | | | | 0.2 | |
| Kojic acid | Kojic Acid | | 0.5 | | | | | | | | 1.0 |
| Liquorice extract | | | | | | | 2.0 | | | | |
| Mg ascorbyl | Magnesium Ascorbyl- | | | | | | | | | 3.0 | |
| phosphate | phosphate | | | | | | | | | | |
| Niacinamide | | | | 1.0 | | | | | | | |
| Soya extract | | | 1.0 | | | | | | | | 1.0 |
| Oil components | | | | | | | | | | | |
| Abil 100 ® | Dimethicone | 1.0 | 0.3 | | | | 1.0 | | | 2.0 | 0.3 |
| (Goldschmidt) | | | | | | | | | | | |
| Cetiol OE (Cognis) | Dicaprylyl Ether | | | | | | 3.0 | | | | |
| Corapan TQ ® | Diethylhexyl 2,6- | | 2.0 | | | | | | | | |
| (Symrise) | Naphthalate | | | | | | | | | | |
| Dragoxat EH | Ethylhexyl | | | 1.0 | 1.0 | | | | | | |
| (Symrise) | Ethylhexanoate | | | | | | | | | | |
| Isoadipate (Symrise) | Diisopropyl Adipate | | | | 1.0 | | | | | | |
| Isopropyl myristate | Isopropyl Myristate | | | | | | | | | | 4.0 |
| (Symrise) | | | | | | | | | | | |
| Isodragol (Symrise) | Triisononanoin | | | | | | | | | 6.0 | |
| Neutral oil (Symrise) | Caprylic/Capric/ | 4.0 | 2.0 | | | 2.0 | | | | | |
| | Triglyceride | | | | | | | | | | |
| Paraffin oil | Mineral Oil | | | | | | | | | | 4.0 |
| PCL Liquid 100 | Cetearyl Octanoate | 4.0 | 3.0 | | | | | | 3.0 | | |
| (Symrise) | | | | | | | | | | | |
| Tegosoft TN ® | C12-C15 Alkyl Benzoate | | | | 4.0 | 2.0 | | | | | |
| (Goldschmidt) | | | | | | | | | | | |
| Further ingredients | | | | | | | | | | | |
| Arlypon F | Laureth-2 | | | | | | | 2.0 | | | |
| alpha-Bisabolol | Bisabolol | | 0.1 | | 0.2 | 0.1 | 0.1 | 0.1 | | | |
| (Symrise) | | | | | | | | | | | |
| 1,3-Butylene glycol | 1,3-Butylene Glycol | | 3.0 | | | | | | | | |
| Carbopol 2050 ® | Carbomer | | 0.2 | | 0.5 | 0.1 | | | | | |
| (B. F. Goodrich) | | | | | | | | | | | |
| Ceramid Bio 391 | Ceramide | 0.3 | | | | | | | | | 0.1 |
| (Symrise) | | | | | | | | | | | |
| Citric Acid | Citric Acid | | | | | | | | 0.1 | 0.3 | |
| Copherol 1250 ® | Tocopherol Acetate | | 0.5 | | 0.5 | 0.5 | 0.5 | | | | |
| (Cognis) | | | | | | | | | | | |
| Crinipan ® AD | Climbazole | | | | | | | 0.5 | | | |
| (Symrise) | | | | | | | | | | | |
| Dehyquart SP | Quaternium-52 | | | | | | | | 0.5 | | |
| Dehyton K | Cocamidopropyl Betaine | | | | | | | 12.0 | | | |
| Dow Corning ® 193 | Dimethicone Polyol | | | 1.0 | | | | | | | |
| (Dow Corning) | | | | | | | | | | | |
| D-Panthenol (BASF) | Panthenol | | | 0.5 | | | 0.5 | 0.4 | | | |
| Dragocid Liquid | Phenoxyethanol (and) | 0.8 | | 0.8 | 0.8 | 0.8 | 0.8 | 0.5 | 0.8 | 0.8 | |
| (Symrise) | Methylparaben (and) | | | | | | | | | | |
| | Ethylparaben (and) | | | | | | | | | | |
| | Butylparaben (and) | | | | | | | | | | |
| | Propylparaben (and) | | | | | | | | | | |
| | Isobutylparaben | | | | | | | | | | |
| Dragophos S | Sodium Dihydroxycetyl | | | | | | | | | 2.0 | |
| (Symrise) | Phosphate | | | | | | | | | | |
| Dracorin CE | Glyceryl Stearate/Citrate | | | | | | | | | 1.0 | |
| Dracorin GMS | Glyceryl Stearate | 2.0 | 2.0 | | | 2.0 | | | | 2.0 | 3.0 |
| (Symrise) | | | | | | | | | | | |
| Dracorin 100 s.e. P | Glyceryl Stearate, PEG- | 3.0 | | | | | | | | | 8.0 |
| (Symrise) | 100 Stearate | | | | | | | | | | |
| Edeta BD ® (BASF) | Disodium EDTA | 0.1 | 0.1 | | 0.1 | 0.1 | 0.1 | | | | |
| Emulgin B2 ® | Ceteareth-20 | 1.0 | | | | | | | 0.7 | | |
| (Cognis) | | | | | | | | | | | |
| Emulsiphos | Potassium | | 1.5 | | | 1.5 | | | | | |
| (Symrise) | Cetylphosphate, | | | | | | | | | | |
| | Hydrogenated Palm | | | | | | | | | | |
| | Glycerides | | | | | | | | | | |
| Ethanol (96%) | Ethyl Alcohol | | | 13.0 | 5.0 | | | | | | |
| Extrapon Aloe Vera | | | | 1.0 | | | | | | | |
| (Symrise) | | | | | | | | | | | |
| Extrapon Kamille | | | | 1.0 | | | | | | | |
| (Symrise) | | | | | | | | | | | |
| Extrapon Hamamelis | | | | 1.0 | | | | | | | |
| (Symrise) | | | | | | | | | | | |
| Frescolat ML | Menthyl Lactate | | | | 0.5 | | | | | | |
| Glycerol 99% | Glycerin | 3.0 | | 4.5 | | 3.0 | 4.0 | | | | |
| Hydrolite-5 | Pentylene Glycol | | | | 4.5 | | 5.0 | | | 3.0 | |
| (Symrise) | | | | | | | | | | | |
| Keltrol T ® (Kelco) | Xanthan Gum | | 0.2 | 0.2 | 0.3 | | | | | | |
| Lanette E ® (Cognis) | Sodium Cetearyl Sulfate | | 0.7 | | | | | | | | |
| Lanette O ® (Cognis) | Cetaeryl Alcohol | 1.1 | | | | | | | 2.5 | | |
| Lanette 16 ® | Cetyl Alcohol | | 1.2 | | | 0.5 | | | | | 2.0 |
| (Cognis) | | | | | | | | | | | |
| Lanette 18 (Care | Stearyl Alcohol | | | | | | | | | 4.5 | |
| Chemicals) | | | | | | | | | | | |
| Lara Care A-200 | Galactoarabinan | | | | | | 0.2 | | | | |
| (Rahn) | | | | | | | | | | | |
| NaOH 10% aq. | Sodium Hydroxide | | 2.8 | | 2.2 | 2.9 | 0.6 | | | | 0.2 |
| solution | | | | | | | | | | | |
| Natrosol 250 HHR | Hydroxymethyl Cellulose | 0.3 | | | | | | | | | |
| (Aqualon) | | | | | | | | | | | |
| Neo Heliopan ® AP | Disodium Phenyl- | | 22.0 | | | | | | | | |
| (Symrise), 15% as | dibenzimidazole Tetra- | | | | | | | | | | |
| sodium salt | sulfonate | | | | | | | | | | |
| Neo Heliopan ® AP | Disodium Phenyl- | | | 22.0 | | | | | | | |
| (Symrise), 10% aq. | dibenzimidazole Tetra- | | | | | | | | | | |
| solution neutralized | sulphonate | | | | | | | | | | |
| with NaOH | | | | | | | | | | | |
| Neo Heliopan ® AV | Ethylhexyl Methoxy- | | | | 5.0 | 6.0 | 2.0 | | | | |
| (Symrise) | cinnamate | | | | | | | | | | |
| Neo Heliopan ® BB | Benzophenone-3 | 1.0 | | | | | | | | | |
| (Symrise) | | | | | | | | | | | |
| Neo Heliopan ® 303 | Octocrylene | 7.0 | | | | | | | | | |
| (Symrise) | | | | | | | | | | | |
| Neo Heliopan ® 357 | Butyl Methoxydibenzoyl- | | 2.0 | | 1.5 | 1.5 | 1.5 | 0.5 | 0.5 | | |
| (Symrise) | methane | | | | | | | | | | |
| Neo Heliopan ® | Isoamyl p- | | | | 5.0 | | 6.0 | | 2.0 | | |
| E 1000 (Symrise) | Methoxycinnamate | | | | | | | | | | |
| Neo Heliopan ® | Homosalate | | 5.0 | | | | | | | | |
| HMS (Symrise) | | | | | | | | | | | |
| Neo Heliopan ® | Phenylbenzimidazole | | | 33.3 | 10.0 | 13.3 | | 3.3 | | | |
| Hydro | Sulfonic Acid | | | | | | | | | | |
| (15% aq. solution | | | | | | | | | | | |
| neutralized with | | | | | | | | | | | |
| NaOH) (Symrise) | | | | | | | | | | | |
| Neo Heliopan ® MA | Menthyl Anthranilate | 3.0 | | | | | | | | | |
| (Symrise) | | | | | | | | | | | |
| Neo Heliopan ® | 4-Methylbenzylidene | | | | 2.0 | 4.0 | 3.0 | | | | |
| MBC (Symrise) | Camphor | | | | | | | | | | |
| Neo Heliopan ® OS | Ethylhexyl Salicylate | 1.0 | | | | | | | | | |
| (Symrise) | | | | | | | | | | | |
| Neo PCL wssl. N | Trideceth-9, PEG-5 | | | 1.0 | 1.5 | | | 1.5 | | | |
| (Symrise) | Ethylhexanoate | | | | | | | | | | |
| Perfume oil | Perfume (Fragrance) | 0.3 | 0.3 | 0.3 | 0.3 | 0.4 | 0.2 | 0.5 | 0.4 | 0.3 | 0.3 |
| (Symrise) | | | | | | | | | | | |
| Pemulen TR 2 | Acrylates/C10-30 Alkyl | | | | | | 0.2 | | | | |
| (Novion) | Acrylate Crosspolymer | | | | | | | | | | |
| Polymer JR 400 | Polyquaternium-10 | | | | | | | 0.4 | | | |
| 1,2-Propylene glycol | Propylene Glycol | | | | | | | | | | 5.0 |
| Simagel M | Quaternium-18 Hectorite | 1.0 | | | | | | | | | |
| Solubilizer (Symrise) | PEG 40 Hydrogenated | | | | | | | 3.0 | | | |
| | Castor Oil, Trideceth-9, | | | | | | | | | | |
| | Propylene Glycol, Water | | | | | | | | | | |
| Symdiol 68 | 1,2-Hexanediol, | | 0.5 | | | | | | | | 1.0 |
| | Caprylylglycol | | | | | | | | | | |
| Texapon N 70 | Sodium Laureth Sulfate | | | | | | 0.5 | | | | |
| (Cognis) | | | | | | | | | | | |
| Texapon NSO BZ | Sodium Laureth Sulfate | | | | | | | 27.0 | | | |
| (Cognis) | | | | | | | | | | | |
| Zink Oxide neutral | Zinc Oxide | 5.0 | | | | | | | | | |
| (Symrise) | | | | | | | | | | | |
| Veegum ultra ® | Magnesium Aluminium | 1.0 | | | | | | | | | |
| (Vanderbilt) | Sulfate | | | | | | | | | | |
| Vitamin A Palmitate | Retinyl Palmitate | | | | | 0.1 | | | | 0.1 | |
| Witch Hazel | Hamamelis Virginiana | | | | | | | 1.0 | | | |
| Distillate (Symrise) | (Witch Hazel) | | | | | | | | | | |
| Water, dist. | Aqua (Water) | ad | ad | ad | ad | ad | ad | ad | ad | ad | ad |
| | | 100 | 100 | 100 | 100 | 100 | 100 | 100 | 100 | 100 | 100 |

Preparation 1: “Oil-in-water” emulsion with UV-A/B-broadband protection
Preparation 2: “Oil-in-water” emulsion with UV-A/B-broadband protection
Preparation 3: Sun spray with UV-A/B-broadband protection with low oil content
Preparation 4: Skin-lightening balm with UV-A/UV-B protection
Preparation 5: Skin-lightening aerosol foam with UV-B/UV-A protection
Preparation 6: Skin-lightening non-aerosol foam
Preparation 7: Shampoo with skin-lightening properties
Preparation 8: Skin-lightening hair conditioner with UV-B/UV-A protection
Preparation 9: Skin-lightening moisturizing cream O/W
Preparation 10: Skin-lightening face cream O/W

### Specific Embodiments

In specific embodiment one, the invention comprises a cosmetic and/or pharmaceutical preparation, comprising a mixture comprising or consisting of
a) a tyrosinase-inhibiting amount of one or more compounds of the formula 1:

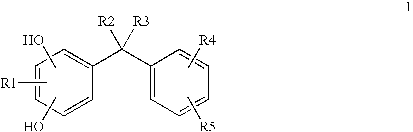

wherein:
   R1 is hydrogen, methyl, straight-chain or branched alkyl having 2-4 C atoms, OH or halogen,
   R2 is hydrogen, methyl or straight-chain or branched alkyl having 2-5 C atoms,
   R3 is methyl or straight-chain or branched alkyl having 2-5 C atoms, and
   R4 and R5 are, independently of one another hydrogen, methyl, straight-chain or branched alkyl having 2-5 C atoms, OH or halogen and
b) a skin- and/or hair lightening and/or senile keratosis-reducing amount of one or more compounds selected from the group consisting of (i) chelating agents, (ii) phenolic derivatives, plant extracts with an amount of phenol derivatives and (iii) organic acid derivatives.

In specific embodiment two, the invention comprises a preparation according to specific embodiment one, wherein R2 is methyl for the or one of the compounds of formula 1.

In specific embodiment three, the invention comprises a preparation according to specific embodiment one or two, wherein the or one of the compounds of formula 1 is styrylresorcinol and/or 4-butylresorcinol.

In specific embodiment four, the invention comprises a preparation according to any of specific embodiments one to three, wherein the or one of the
(i) chelating agents are selected from the group consisting of kojic acid, EDTA, hinokitiol, tropolone, ascorbic acid, lactic acid, salicylic acid, glycolic acid, citric acid and malic acid, and/or
(ii) phenolic derivatives and plant extracts comprising an amount of phenolic derivatives are selected from the group consisting of arbutin, hydroquinone, resorcinol, 4-butyl resorcinol, bearberry extract (Arctostaphylos uva-ursi), pinus extract (Pinus sylvestris), Mulberry extract (Morus alba), soybean extract (Glycine max.), artocarpus extract (Artocarpus incisus) and licorice extract (Glycyrrhiza glabra), and/or
(iii) organic acid derivatives compounds are selected from the group consisting of azelaic, 9-octadecenoic acid, alpha lipoic acid, retinoic acid, niacinamide and undecylenoyl phenylalanine.

In specific embodiment five, the invention comprises a preparation according to any of specific embodiments one to four, wherein the or one of the
(i) chelating agents are selected from the group consisting of kojic acid, EDTA, ascorbic acid, lactic acid and salicylic acid, and/or
(ii) phenolic derivatives are selected from the group consisting of arbutin, hydroquinone and 4-butyl resorcinol, and plant extracts are selected from the group consisting of bearberry extract (Arctostaphylos uva-ursi), Mulberry extract (Morus alba), artocarpus extract (Artocarpus incisus) and licorice extract (Glycyrrhiza glabra), and/or
(iii) organic acid derivatives are selected from the group consisting of azelaic, 9-octadecenoic acid, niacinamide and undecylenoyl phenylalanine.

In specific embodiment six, the invention comprises a preparation according to any of specific embodiments one to five, wherein the preparation is in the form of an O/W emulsion.

In specific embodiment seven, the invention comprises a preparation according to any of specific embodiments one to six, furthermore comprising at least one UV filter, preferably at least in an amount which is capable of preventing discolouration of the preparation caused by (sun)light.

In specific embodiment eight, the invention comprises a preparation according to any of specific embodiments one to seven, furthermore comprising a total amount of UV filters and/or inorganic pigments such that the preparation according to the invention has a sunscreen factor of greater than or equal to 2, preferably greater than or equal to 5.

In specific embodiment nine, the invention comprises a preparation according to any of specific embodiments one to eight, furthermore comprising a cooling active compound in an amount sufficient to achieve a skin-cooling effect.

In specific embodiment ten, the invention comprises a preparation according to any of specific embodiments one to nine, furthermore comprising one or more compounds for care and/or cleansing of (a) skin and/or (b) hair.

In specific embodiment eleven, the invention comprises a preparation according to any of specific embodiments one to ten, furthermore comprising a sensorially active amount of one or more odoriferous substances.

In specific embodiment twelve, the invention comprises a use of a mixture comprising or consisting of
a) a tyrosinase-inhibiting amount of one or more compounds of the formula 1:

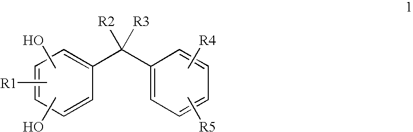

wherein:
   R1 is hydrogen, methyl, straight-chain or branched alkyl having 2-4 C atoms, OH or halogen,
   R2 is hydrogen, methyl or straight-chain or branched alkyl having 2-5 C atoms,
   R3 is methyl or straight-chain or branched alkyl having 2-5 C atoms, and
   R4 and R5 are, independently of one another hydrogen, methyl, straight-chain or branched alkyl having 2-5 C atoms, OH or halogen and
b) a skin- and/or hair lightening and/or senile keratosis-reducing amount of one or more compounds selected from the group consisting of (i) chelating agents, (ii) phenolic derivatives, plant extracts with an amount of phenolic derivatives and (iii) organic acid derivatives
in the manufacture of a cosmetic and/or pharmaceutical preparation for lightening skin and/or reducing senile keratosis.

In specific embodiment thirteen, the invention comprises a use of a preparation according to any of specific embodiments one to eleven for hair lightening.

In specific embodiment fourteen, the invention comprises a method for lightening skin and/or hair and/or for reducing senile keratosis, comprising or consisting of the step:
application of a preparation according to any of specific embodiments one to eleven to skin and/or hair.

In specific embodiment fifteen, the invention comprises a process for the production of a preparation for lightening skin and/or hair and/or for reducing senile keratosis, comprising or consisting of the following steps:
a) providing one or more compounds of the formula 1

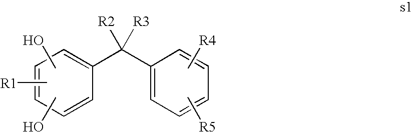

wherein:
   R1 is hydrogen, methyl, straight-chain or branched alkyl having 2-4 C atoms, OH or halogen,
   R2 is hydrogen, methyl or straight-chain or branched alkyl having 2-5 C atoms,
   R3 is methyl or straight-chain or branched alkyl having 2-5 C atoms, and
   R4 and R5 are, independently of one another hydrogen, methyl, straight-chain or branched alkyl having 2-5 C atoms, OH or halogen,
b) providing one or more compounds selected from the group of (i) chelating agents, (ii) phenolic derivatives, plant extracts with an amount of phenolic derivatives and (iii) organic acid derivatives,
c) providing one or more further compounds and
d) mixing one or more compounds provided in step d) and one or more compounds provided in step e) together with one or more compounds provided in step f) to form a preparation according to any of specific embodiments one to eleven.

It is claimed:

1. A cosmetic and/or pharmaceutical preparation, comprising a mixture comprising
a) a tyrosinase-inhibiting amount of styrylresorcinol in an amount of 0.1 to 2 wt. % based on the finished cosmetic and/or pharmaceutical preparation, and
b) a skin- and/or hair lightening and/or senile keratosis-reducing amount of one or more compounds selected from the group consisting of
(i) chelating agents selected from the group consisting of
Kojic acid in an amount of 0.5-5 wt % and in a ratio to styrylresorcinol of 1:4 to 50:1 based on the finished cosmetic and/or pharmaceutical preparation,
EDTA in an amount of 0.02-0.5 wt % and in a ratio to styrylresorcinol of 1:100 to 5:1 based on the finished cosmetic and/or pharmaceutical preparation,
Hinokitiol in an amount of 0.1-1 wt % and in a ratio to styrylresorcinol of 1:20 to 10:1 based on the finished cosmetic and/or pharmaceutical preparation,
Tropolone in an amount of 0.05-1 wt % and in a ratio to styrylresorcinol of 1:40 to 10:1 based on the finished cosmetic and/or pharmaceutical preparation,
Ascorbic acid in an amount of 0.5-5 wt % and in a ratio to styrylresorcinol of 1:4 to 50:1 based on the finished cosmetic and/or pharmaceutical preparation,
Lactic acid in an amount of 0.2-5 wt % and in a ratio to styrylresorcinol of 1:10 to 50:1 based on the finished cosmetic and/or pharmaceutical preparation,
Salicylic acid in an amount of 0.2-5 wt % and in a ratio to styrylresorcinol of 1:10 to 50:1 based on the finished cosmetic and/or pharmaceutical preparation,
Glycolic acid in an amount of 0.2-5 wt % and in a ratio to styrylresorcinol of 1:10 to 50:1 based on the finished cosmetic and/or pharmaceutical preparation,
Citric acid in an amount of 0.2-5 wt % and in a ratio to styrylresorcinol of 1:10 to 50:1 based on the finished cosmetic and/or pharmaceutical preparation and
Malic acid in an amount of 0.2-5 wt % and in a ratio to styrylresorcinol of 1:10 to 50:1 based on the finished cosmetic and/or pharmaceutical preparation,
(ii) phenolic derivatives and plant extracts comprising phenol derivatives selected from the group consisting of:
Arbutine in an amount of 0.5-5 wt % and in a ratio to styrylresorcinol of 1:4 to 50:1 based on the finished cosmetic and/or pharmaceutical preparation,
Hydroquinone in an amount of 0.2-4 wt % and in a ratio to styrylresorcinol of 1:10 to 40:1 based on the finished cosmetic and/or pharmaceutical preparation,
Resorcinol in an amount of 0.5-5 wt % and in a ratio to styrylresorcinol of 1:4 to 50:1 based on the finished cosmetic and/or pharmaceutical preparation,
4-Butylresorcinol in an amount of 0.5-5 wt % and in a ratio to styrylresorcinol of 1:4 to 50:1 based on the finished cosmetic and/or pharmaceutical preparation,
Bearberry extract in an amount of 0.5-5 wt % and in a ratio to styrylresorcinol of 1:4 to 50:1 based on the finished cosmetic and/or pharmaceutical preparation,
Pinus extract in an amount of 0.5-5 wt % and in a ratio to styrylresorcinol of 1:4 to 50:1 based on the finished cosmetic and/or pharmaceutical preparation,
Mulberry extract in an amount of 0.5-5 wt % and in a ratio to styrylresorcinol of 1:4 to 50:1 based on the finished cosmetic and/or pharmaceutical preparation,
Soybean extract in an amount of 0.5-5 wt % and in a ratio to styrylresorcinol of 1:4 to 50:1 based on the finished cosmetic and/or pharmaceutical preparation,
Artocarpus extract in an amount of 0.5-5 wt % and in a ratio to styrylresorcinol of 1:4 to 50:1 based on the finished cosmetic and/or pharmaceutical preparation and
Licorice extract in an amount of 0.5-5 wt % and in a ratio to styrylresorcinol of 1:4 to 50:1 based on the finished cosmetic and/or pharmaceutical preparation and/or
(iii) organic acid derivatives selected of the group consisting of:
Azelaic acid In an amount of 0.5-5 wt % and in a ratio to styrylresorcinol of 1:4 to 50:1 based on the finished cosmetic and/or pharmaceutical preparation,
9-Octadeceneoic acid in an amount of 0.5-5 wt % and in a ratio to styrylresorcinol of 1:4 to 50:1 based on the finished cosmetic and/or pharmaceutical preparation,
alpha-Lipoic acid in an amount of 0.2-1 wt % and in a ratio to styrylresorcinol of 1:10 to 10:1 based on the finished cosmetic and/or pharmaceutical preparation,
Retinoic acid in an amount of 0.01-0.1 wt % and in a ratio to styrylresorcinol of 1:200 to 1:1 based on the finished cosmetic and/or pharmaceutical preparation,
Niacinamide in an amount of 0.5-5 wt % and in a ratio to styrylresorcinol of 1:4 to 50:1 based on the finished cosmetic and/or pharmaceutical preparation and
Undecylenoyl Phenylalanine in an amount of 0.5-5 wt % and in a ratio to styrylresorcinol of 1:4 to 50:1 based on the finished cosmetic and/or pharmaceutical preparation.

2. The preparation according to claim 1, wherein the preparation is in the form of an O/W emulsion.

3. The preparation according to claim 1, furthermore comprising at least one UV filter.

4. The preparation according to claim 1, furthermore comprising a total amount of UV filters and/or inorganic pigments such that the preparation according to the invention has a sunscreen factor of greater than or equal to 2.

5. The preparation according to claim 1, furthermore comprising a cooling active compound in an amount sufficient to achieve a skin-cooling effect.

6. The preparation according to claim 1, furthermore comprising one or more compounds for care and/or cleansing of (a) skin and/or (b) hair.

7. The preparation according to claim 1, furthermore comprising a sensorially active amount of one or more odoriferous substances.

8. A method for manufacture of a cosmetic and/or pharmaceutical composition for lightening skin and/or reducing senile keratosis comprising mixing
a) a tyrosinase-inhibiting amount of styrylresorcinol in an amount of 0.1 to 2 wt. % based on the finished cosmetic and/or pharmaceutical preparation, and
b) a skin- and/or hair lightening and/or senile keratosis-reducing amount of one or more compounds selected from the group consisting of
(i) chelating agents selected from the group consisting of
Kojic acid in an amount of 0.5-5 wt % and in a ratio to styrylresorcinol of 1:4 to 50:1 based on the finished cosmetic and/or pharmaceutical preparation,
EDTA in an amount of 0.02-0.5 wt % and in a ratio to styrylresorcinol of 1:100 to 5:1 based on the finished cosmetic and/or pharmaceutical preparation,
Hinokitiol in an amount of 0.1-1 wt % and in a ratio to styrylresorcinol of 1:20 to 10:1 based on the finished cosmetic and/or pharmaceutical preparation,
Tropolone in an amount of 0.05-1 wt % and in a ratio to styrylresorcinol of 1:40 to 10:1 based on the finished cosmetic and/or pharmaceutical preparation,
Ascorbic acid in an amount of 0.5-5 wt % and in a ratio to styrylresorcinol of 1:4 to 50:1 based on the finished cosmetic and/or pharmaceutical preparation,
Lactic acid In an amount of 0.2-5 wt % and in a ratio to styrylresorcinol of 1:10 to 50:1 based on the finished cosmetic and/or pharmaceutical preparation,
Salicylic acid in an amount of 0.2-5 wt % and in a ratio to styrylresorcinol of 1:10 to 50:1 based on the finished cosmetic and/or pharmaceutical preparation,
Glycolic acid in an amount of 0.2-5 wt % and in a ratio to styrylresorcinol of 1:10 to 50:1 based on the finished cosmetic and/or pharmaceutical preparation,
Citric acid in an amount of 0.2-5 wt % and in a ratio to styrylresorcinol of 1:10 to 50:1 based on the finished cosmetic and/or pharmaceutical preparation and
Malic acid in an amount of 0.2-5 wt % and in a ratio to styrylresorcinol of 1:10 to 50:1 based on the finished cosmetic and/or pharmaceutical preparation,
(ii) phenolic derivatives and plant extracts comprising phenol derivatives selected from the group consisting of:
Arbutine in an amount of 0.5-5 wt % and In a ratio to styrylresorcinol of 1:4 to 50:1 based on the finished cosmetic and/or pharmaceutical preparation,
Hydroquinone in an amount of 0.2-4 wt % and in a ratio to styrylresorcinol of 1:10 to 40:1 based on the finished cosmetic and/or pharmaceutical preparation,
Resorcinol in an amount of 0.5-5 wt % and in a ratio to styrylresorcinol of 1:4 to 50:1 based on the finished cosmetic and/or pharmaceutical preparation,
4-Butylresorcinol in an amount of 0.5-5 wt % and in a ratio to styrylresorcinol of 1:4 to 50:1 based on the finished cosmetic and/or pharmaceutical preparation,
Bearberry extract in an amount of 0.5-5 wt % and in a ratio to styrylresorcinol of 1:4 to 50:1 based on the finished cosmetic and/or pharmaceutical preparation,
Pinus extract in an amount of 0.5-5 wt % and in a ratio to styrylresorcinol of 1:4 to 50:1 based on the finished cosmetic and/or pharmaceutical preparation,
Mulberry extract in an amount of 0.5-5 wt % and in a ratio to styrylresorcinol of 1:4 to 50:1 based on the finished cosmetic and/or pharmaceutical preparation,
Soybean extract in an amount of 0.5-5 wt % and in a ratio to styrylresorcinol of 1:4 to 50:1 based on the finished cosmetic and/or pharmaceutical preparation,
Artocarpus extract in an amount of 0.5-5 wt % and in a ratio to styrylresorcinol of 1:4 to 50:1 based on the finished cosmetic and/or pharmaceutical preparation and
Licorice extract in an amount of 0.5-5 wt % and in a ratio to styrylresorcinol of 1.4 to 50:1 based on the finished cosmetic and/or pharmaceutical preparation and/or
(iii) organic acid derivatives selected of the group consisting of:
Azelaic acid in an amount of 0.5-5 wt % and in a ratio to styrylresorcinol of 1:4 to 50:1 based on the finished cosmetic and/or pharmaceutical preparation,
9-Octadeceneoic acid in an amount of 0.5-5 wt % and in a ratio to styrylresorcinol of 1:4 to 50:1 based on the finished cosmetic and/or pharmaceutical preparation,
alpha-Lipoic acid in an amount of 0.2-1 wt % and in a ratio to styrylresorcinol of 1:10 to 10:1 based on the finished cosmetic and/or pharmaceutical preparation,
Retinoic acid in an amount of 0.01-0.1 wt % and in a ratio to styrylresorcinol of 1:200 to 1:1 based on the finished cosmetic and/or pharmaceutical preparation,
Niacinamide in an amount of 0.5-5 wt % and in a ratio to styrylresorcinol of 1:4 to 50:1 based on the finished cosmetic and/or pharmaceutical preparation and
Undecylenoyl Phenylalanine in an amount of 0.5-5 wt % and in a ratio to styrylresorcinol of 1:4 to 50:1 based on the finished cosmetic and/or pharmaceutical preparation

9. A method for hair lightening comprising application of a preparation according to claim 1.

10. A method for lightening skin and/or hair and/or for reducing senile keratosis, comprising:
c) application of a preparation according to claim 1 to skin and/or hair.

11. A process for the production of a preparation for lightening skin and/or hair and/or for reducing senile keratosis, comprising providing:
d) a tyrosinase-inhibiting amount of styrylresorcinol in an amount of 0.1 to 2 wt. % based on the finished cosmetic and/or pharmaceutical preparation,
e) a skin- and/or hair lightening and/or senile keratosis-reducing amount of one or more compounds selected from the group consisting of
(i) chelating agents selected from the group consisting of
Kojic acid in an amount of 0.5-5 wt % and in a ratio to styrylresorcinol of 1:4 to 50:1 based on the finished cosmetic and/or pharmaceutical preparation,
EDTA in an amount of 0.02-0.5 wt % and in a ratio to styrylresorcinol of 1:100 to 5:1 based on the finished cosmetic and/or pharmaceutical preparation,
Hinokitiol in an amount of 0.1-1 wt % and in a ratio to styrylresorcinol of 1:20 to 10:1 based on the finished cosmetic and/or pharmaceutical preparation,
Tropolone in an amount of 0.05-1 wt % and in a ratio to styrylresorcinol of 1:40 to 10:1 based on the finished cosmetic and/or pharmaceutical preparation,
Ascorbic acid in an amount of 0.5-5 wt % and in a ratio to styrylresorcinol of 1:4 to 501 based on the finished cosmetic and/or pharmaceutical preparation,
Lactic acid in an amount of 0.2-5 wt % and in a ratio to styrylresorcinol of 1:10 to 50:1 based on the finished cosmetic and/or pharmaceutical preparation,
Salicylic acid in an amount of 0.2-5 wt % and in a ratio to styrylresorcinol of 1:10 to 50:1 based on the finished cosmetic and/or pharmaceutical preparation,
Glycolic acid in an amount of 0.2-5 wt % and in a ratio to styrylresorcinol of 1:10 to 50:1 based on the finished cosmetic and/or pharmaceutical preparation,
Citric acid in an amount of 0.2-5 wt % and in a ratio to styrylresorcinol of 1:10 to 50:1 based on the finished cosmetic and/or pharmaceutical preparation and
Malic acid in an amount of 0.2-5 wt % and in a ratio to styrylresorcinol of 1:10 to 50:1 based on the finished cosmetic and/or pharmaceutical preparation,
(ii) phenolic derivatives and plant extracts comprising phenol derivatives selected from the group consisting of:
Arbutine in an amount of 0.5-5 wt % and in a ratio to styrylresorcinol of 1:4 to 50:1 based on the finished cosmetic and/or pharmaceutical preparation,
Hydroquinone in an amount of 0.2-4 wt % and in a ratio to styrylresorcinol of 1:10 to 40:1 based on the finished cosmetic and/or pharmaceutical preparation,
Resorcinol in an amount of 0.5-5 wt % and in a ratio to styrylresorcinol of 1:4 to 50:1 based on the finished cosmetic and/or pharmaceutical preparation,
4-Butylresorcinol in an amount of 0.5-5 wt % and in a ratio to styrylresorcinol of 1:4 to 50:1 based on the finished cosmetic and/or pharmaceutical preparation,
Bearberry extract in an amount of 0.5-5 wt % and in a ratio to styrylresorcinol of 1:4 to 50:1 based on the finished cosmetic and/or pharmaceutical preparation,
Pinus extract in an amount of 0.5-5 wt % and in a ratio to styrylresorcinol of 1:4 to 50:1 based on the finished cosmetic and/or pharmaceutical preparation,
Mulberry extract in an amount of 0.5-5 wt % and in a ratio to styrylresorcinol of 1:4 to 50:1 based on the finished cosmetic and/or pharmaceutical preparation,
Soybean extract in an amount of 0.5-5 wt % and in a ratio to styrylresorcinol of 1:4 to 50:1 based on the finished cosmetic and/or pharmaceutical preparation,
Artocarpus extract in an amount of 0.5-5 wt % and in a ratio to styrylresorcinol of 1:4 to 50:1 based on the finished cosmetic and/or pharmaceutical preparation and
Licorice extract in an amount of 0.5-5 wt % and in a ratio to styrylresorcinol of 1:4 to 50:1 based on the finished cosmetic and/or pharmaceutical preparation and/or
(iii) organic acid derivatives selected of the group consisting of:
Azelaic acid in an amount of 0.5-5 wt % and in a ratio to styrylresorcinol of 1:4 to 50:1 based on the finished cosmetic and/or pharmaceutical preparation,
9-Octadeceneoic acid in an amount of 0.5-5 wt % and in a ratio to styrylresorcinol of 1:4 to 50:1 based on the finished cosmetic and/or pharmaceutical preparation,
alpha-Lipoic acid in an amount of 0.2-1 wt % and in a ratio to styrylresorcinol of 1:10 to 10:1 based on the finished cosmetic and/or pharmaceutical preparation,
Retinoic acid in an amount of 0.01-0.1 wt % and in a ratio to styrylresorcinol of 1:200 to 1:1 based on the finished cosmetic and/or pharmaceutical preparation,
Niacinamide in an amount of 0.5-5 wt % and in a ratio to styrylresorcinol of 1:4 to 50:1 based on the finished cosmetic and/or pharmaceutical preparation and
Undecylenoyl Phenylalanine in an amount of 0.5-5 wt % and in a ratio to styrylresorcinol of 1:4 to 50:1 based on the finished cosmetic and/or pharmaceutical preparation
(f) providing one or more further compounds and
(g) mixing one or more compounds provided in step d) and one or more compounds provided in step e) together with one or more compounds provided in step t) to form a preparation according to claim 1.

12. The preparation according to claim 3, wherein the UV filter is present in at least in an amount which is capable of preventing discoloration of the preparation caused by (sun)light.

13. The preparation according to claim 4, wherein the preparation has a sunscreen factor greater than or equal to 5.
